(19) United States
(12) Patent Application Publication
(10) Pub. No.: US 2026/0200982 A1
(43) Pub. Date: Jul. 16, 2026

(54) HER RECEPTOR DERIVED PEPTIDES AND METHODS OF USE THEREOF

(71) Applicant: Ramot at Tel-Aviv University Ltd., Tel-Aviv (IL)

(72) Inventor: Ronit PINKAS-KRAMARSKI, Tel-Aviv (IL)

(73) Assignee: Ramot at Tel-Aviv University Ltd., Tel-Aviv (IL)

(21) Appl. No.: 19/130,949

(22) PCT Filed: Nov. 2, 2023
(86) PCT No.: PCT/IL2023/051130

§ 371 Date: May 18, 2025

Related U.S. Application Data
(60) Provisional application No. 63/426,381, filed on Nov. 18, 2022.

(51) Int. Cl. C07K 7/08; A61K 38/19; A61P 35/00

(52) U.S. Cl. CPC C07K 7/08; A61K 38/195; A61P 35/00; C07K 2319/09; C07K 2319/10

(57) ABSTRACT

HER receptor derived peptides are provided. Accordingly there is provided a composition of matter comprising a peptide comprising an amino acid sequence of a nuclear localization sequence (NLS) of a HER receptor capable of binding a nucleolin polypeptide, the peptide being attached to a cell penetrating moiety. Also provided are polynucleotides encoding the composition of matter, host cells expressing the composition of matter and methods of using same.

### RELATED APPLICATION/S

This application claims the benefit of priority of U.S. Provisional Patent Application No. 63/426,381 filed on Nov. 18, 2022, the contents of which are incorporated herein by reference in their entirety.

### SEQUENCE LISTING STATEMENT

The XML file, entitled 97734 Sequence Listing.xml, created on Nov. 1, 2023, comprising 39,608 bytes, submitted concurrently with the filing of this application is incorporated herein by reference.

### FIELD AND BACKGROUND OF THE INVENTION

The present invention, in some embodiments thereof, relates to HER receptor derived peptides and methods of use thereof.

Growth factors and their transmembrane receptor tyrosine kinases (RTKs) regulate various cellular processes, including proliferation, differentiation, migration and survival. One remarkable example entails a large family of growth factors, all sharing an epidermal growth factor (EGF) motif, and their respective RTKs of the ErbB/HER family. The HER family includes four receptors, the epidermal growth factor receptor, EGFR (ErbB1/HER1), HER2 (c-Neu, ErbB2), HER3 (ErbB3) and HER4 (ErbB4). HER receptors harbor an extracellular domain, followed by a transmembrane domain and an intracellular domain, which provides tyrosine kinase activity.

Ligand induced activation of HER family receptor members lead to homodimerization/heterodimerization, phosphorylation of specific tyrosine residues, and recruitment of several proteins at the intracellular portion of the receptors leading to activation of e.g. the Ras-Raf-MAPK and PI3-K/AKT pathways.

The HER family proteins have been described in involvement of several pathologies, including psoriasis, atherosclerosis, as well as in several types of human cancer. For instance, many tumors of epithelial origins express an increased level of EGFR on their cell surface [Ullrich, A. et al. (1984) Nature 309, 418-425]; and HER2 is overexpressed in many type of cancers, including breast cancer, with approximately 30% of the patients exhibiting high levels of the protein [2]. Consistent with their essential roles in tumor progression, strategies able to interfere with HER functions, such as monoclonal antibodies (mAbs) and tyrosine kinase inhibitors (TKIs), have yielded in the last several decades several oncology drugs. For example, two genetically engineered mAbs to EGFR, cetuximab and panitumumab, are approved for treatment of colorectal cancer; and trastuzumab (Herceptin) that targets HER2 is currently employed routinely in breast cancer therapy. However, many cancers are resistant to HER targeted therapy and many others become resistant following prolonged treatment.

Nucleolin is a multifunctional protein frequently deregulated in cancer cells, where it is highly expressed on the plasma membrane [4, 5]. High nucleolin levels were found to correlate with poor prognosis and increased tumor growth [9]. Nucleolin was shown to directly interact with HER receptors [6] and with Ras [7]. In EGFR, HER2 and Ras-driven cancers (e.g. glioma, breast, prostate and colon cancers), this interaction has been shown to lead to ligand-independent activation of the receptor and to increased cell transformation, both in vitro and in vivo [7-13]. Moreover, an anti-nucleolin G-rich oligonucleotide, GroA (AS1411) was shown to have anti-tumor effects on EGFR and/or Ras overexpressing prostate, colon and glioma cancers and HER2-positive breast cancer cells both in-vitro and in-vivo; an effect which was further augmented by combination with the anti-Ras drug FTS (Salirasib) [8, 9, 12, 13].

### SUMMARY OF THE INVENTION

According to an aspect of some embodiments of the present invention there is provided a composition of matter comprising a peptide comprising an amino acid sequence of a nuclear localization sequence (NLS) of a HER receptor capable of binding a nucleolin polypeptide, the peptide being attached to a cell penetrating moiety.

According to some embodiments of the invention, the cell penetrating moiety is selected from the group consisting of a myristoyl group, a nanoparticle, a liposome, a cell penetrating peptide and poly(alkylene) glycols.

According to some embodiments of the invention, the cell penetrating moiety comprises a myristoyl group.

According to some embodiments of the invention, the cell penetrating moiety is a cell penetrating peptide.

According to some embodiments of the invention, the cell penetrating peptide is selected from the group consisting of TAT, poly R sequence, penetratin, transportan, pIs1, pVEC, MTS, and MAP peptides.

According to some embodiments of the invention, the cell penetrating peptide is selected from the group consisting of TAT and poly R sequence peptides.

According to some embodiments of the invention, the peptide comprising the amino acid sequence of the NLS of a HER receptor is a stapled peptide.

According to some embodiments of the invention, stapling is effected by replacing amino acids residues i and i+4 of the amino acid sequence with S-pentenylalanine (S5); or by replacing amino acids residues i and i+7 of the amino acid sequence with R-octenylalanine (R8) and S-pentenylalanine.

According to an aspect of some embodiments of the present invention there is provided a polynucleotide encoding a peptide comprising an amino acid sequence of a nuclear localization sequence (NLS) of a HER receptor capable of binding a nucleolin polypeptide, the amino acid sequence of the NLS does not exceed 16 amino acids.

According to an aspect of some embodiments of the present invention there is provided a polynucleotide encoding the composition of matter.

According to some embodiments of the invention, the amino acid sequence of the NLS does not exceed 16 amino acids.

According to some embodiments of the invention, the amino acid sequence of the NLS is at least 10 amino acids long.

According to an aspect of some embodiments of the present invention there is provided a nucleic acid construct comprising the polynucleotide and a cis-acting regulatory element for directing expression of the polynucleotide.

According to an aspect of some embodiments of the present invention there is provided a host cell comprising the composition of matter, the polynucleotide or the nucleic acid construct.

According to some embodiments of the invention, the NLS of the HER receptor is an NLS of epidermal growth factor receptor (EGFR).

According to some embodiments of the invention, the peptide comprises SEQ ID NO: 1.

According to some embodiments of the invention, the amino acid sequence is as set forth in SEQ ID NO: 1.

According to some embodiments of the invention, the peptide comprises SEQ ID NO: 18, 19, 20, 21 or 22.

According to some embodiments of the invention, the amino acid sequence is as set forth in SEQ ID NO: 18, 19, 20, 21 or 22.

According to some embodiments of the invention, the peptide comprises SEQ ID NO: 7, 8, 9, 10 or 11.

According to some embodiments of the invention, the amino acid sequence is as set forth in SEQ ID NO: 7, 8, 9, 10 or 11.

According to some embodiments of the invention, the NLS of the HER receptor is an NLS of a receptor selected from the group consisting of HER2, HER3 and HER4.

According to some embodiments of the invention, the peptide comprises SEQ ID NO: 12, 13 or 14.

According to some embodiments of the invention, the amino acid sequence is as set forth in SEQ ID NO: 12, 13 or 14.

According to some embodiments of the invention, the NLS of said HER receptor is an NLS of HER4.

According to some embodiments of the invention, the peptide comprises SEQ ID NO: 14.

According to some embodiments of the invention, the amino acid sequence is as set forth in SEQ ID NO: 14.

According to an aspect of some embodiments of the present invention there is provided a method of treating a disease associated with a HER receptor, the method comprising administering to the subject a therapeutically effective amount of the composition of matter, the polynucleotide, the nucleic acid construct, or the host cell, thereby treating the disease in the subject.

According to an aspect of some embodiments of the present invention there is provided the composition of matter, the polynucleotide, the nucleic acid construct, or the host cell for use in treating a disease associated with a HER receptor.

According to some embodiments of the invention, pathologic cells of the disease overexpress the HER receptor associated with the disease as compared to non-pathologic cells of the same origin.

According to some embodiments of the invention, the HER receptor associated with the disease is epidermal growth factor receptor (EGFR).

According to some embodiments of the invention, pathologic cells of the disease overexpress the nucleolin as compared to non-pathologic cells of the same origin.

According to some embodiments of the invention, pathologic cells of the disease overexpress Ras as compared to non-diseased cells of the same origin.

According to some embodiments of the invention, the method further comprising administering an additional therapeutic agent.

According to some embodiments of the invention, the composition of matter, the polynucleotide, the nucleic acid construct or the host cell for use, further comprising an additional therapeutic agent.

According to some embodiments of the invention, the additional therapeutic agent is specific for a HER receptor, nucleolin and/or Ras.

According to some embodiments of the invention, the additional therapeutic agent is GroA.

According to some embodiments of the invention, the disease is selected from the group consisting of cancer, psoriasis and atherosclerosis.

According to some embodiments of the invention, the disease is cancer.

According to some embodiments of the invention, the cancer is selected from the group consisting of breast cancer, prostate cancer, pancreatic cancer, ovarian cancer, colon cancer and glioma.

According to some embodiments of the invention, the cancer is selected from the group consisting of breast cancer, colon cancer and glioma.

According to some embodiments of the invention, the cancer is selected from the group consisting of colon cancer and pancreatic cancer.

According to an aspect of some embodiments of the present invention there is provided a method of producing a peptide, the method comprising introducing into a host cell the polynucleotide or the nucleic acid construct or culturing the host cell.

According to some embodiments of the invention, the method comprising isolating the peptide.

According to an aspect of some embodiments of the present invention there is provided a method of producing a peptide, the method comprising chemically synthesizing the composition of matter.

According to an aspect of some embodiments of the present invention there is provided a method of producing a peptide, the method comprising chemically synthesizing a peptide comprising an amino acid sequence of a nuclear localization sequence (NLS) of a HER receptor capable of binding a nucleolin polypeptide, the amino acid sequence of the NLS is 16 amino acids long or less.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the chemical structure of the generated peptides: NLS (or NLS1, SEQ ID NO: 1), TAT (SEQ ID NO: 2), TAT-NLS (SEQ ID NO: 3), R-NLS (SEQ ID NO: 4), myristoylated NLS (referred to herein as myr-NLS or myr-NLS1, SEQ ID NO: 5), FITC-tagged myristoylated NLS (referred to herein as myr-NLS-FITC, SEQ ID NO: 6), stapled myristoylated NLS variants (referred to herein as mN6-10, mN7-11, mN8-12, mN11-15 and mN8-15, SEQ ID Nos: 7-11), NLS2 (SEQ ID NO: 12), NLS3 (SEQ ID NO: 13), NLS4 (SEQ ID NO: 14), myristoylated NLS2 (referred to herein as myr-NLS2, SEQ ID NO: 15), myristoylated NLS3 (referred to herein as myr-NLS3, SEQ ID NO: 16) and myristoylated NLS4 (referred to herein as myr-NLS4, SEQ ID NO: 17).

FIGS. 2A-D demonstrate that the TAT-NLS peptide (SEQ ID NO: 3) specifically inhibits viability of cancer cells. FIG. 2A is a bar graph demonstrating cell viability analysis of SKBR3 breast cancer cells, untreated or treated with 80 μM of NLS (SEQ ID NO: 1), TAT (SEQ ID NO: 2) or TAT-NLS (SEQ ID NO: 3) peptides for 72 hours (p-value <0.005; *—compared to untreated; \hat{ }—compared to NLS; #—compared to TAT peptide). FIG. 2B is a bar graph demonstrating the effect of TAT-NLS (SEQ ID NO: 3) treatment on cell viability of either non-cancerous (MCF10A) or cancerous (MCF7 or SKBR3) breast cell lines (p-value <0.05; *—compared to untreated; \hat{ }-compared to TAT peptide). FIG. 2C shows dose-response analysis of the effect of TAT (SEQ ID NO: 2) or TAT-NLS (SEQ IS NO: 3) peptides on viability of breast (SKBR3, MCF7) and colon (DLD-1, HCT-116) cancer cells, following treatment with for 72 hours. Inhibitory concentration (IC-50) was determined for each cell line (p-value <0.05; *—regression analysis; \hat{ }—compared to TAT peptide). FIG. 2D shows time course analysis of the effect of TAT (SEQ ID NO: 2) or TAT-NLS (SEQ IS NO: 3) peptides on viability of breast (SKBR3) and colon (DLD-1, HCT-116) cancer cells, following treatment for the indicated time periods.

FIGS. 3A-B demonstrate that the TAL-NLS peptide (SEQ ID NO: 3) disrupts the ErbB-nucleolin interaction and receptor signaling. FIG. 3A are images of proximity ligation assay (PLA) analysis of ErbB2-nucleolin complexes (red) in SKBR3 cells following treatment with TAT (SEQ ID NO: 2) and TAT-NLS (SEQ ID NO: 3). FIG. 3B demonstrates activation levels of ErbB2 and EGFR following treatment with a TAT (SEQ ID NO: 2) or TAT-NLS (SEQ ID NO: 3) peptide in the indicated cell lines, as determined by western-blot analysis. Upper panel shows representative blots; and lower panels shows quantification analyses (50 μM, 24 hours; p-value <0.05; *—compared to untreated; \hat{ }—compared to TAT peptide).

FIGS. 4A-E demonstrate the inhibitory activity of the R-NLS (SEQ ID NO: 4) and myr-NLS (SEQ ID NO: 5) peptides. FIG. 4A is a bar graph demonstrating the effect of the indicated peptides on SKBR3 cell viability (treatment was induced with 80 μM peptide for 72 hours; p-value <0.005; *—compared to untreated; \hat{ }—compared to TAT-NLS peptide (SEQ ID NO: 3). FIG. 4B shows dose-response analysis of the effect of myr-NLS (SEQ ID NO: 5) on viability of glioma (U87), breast (MCF7 and SKBR3), pancreatic (Panc-1 and MIA Paca), Prostate (LNCaP) and colon (HCT-116 and DLD-1) cancer cells, following treatment for 72 hours. IC-50 was determined for each cell line (p-value <0.05; *—regression analysis). FIG. 4C shows the effect of myr-NLS (SEQ ID NO: 5) on cell viability of cancerous and non cancerous cell lines (MCF7 and SKBR3 upper panel, breast; Rat1-EJ lower panel, fibroblasts transformed with constitutively active Ras) as compared to their respective non-cancerous cells (MCF10A; Rat-1) (p-value <0.005; *—regression analysis; \hat{ }—compared to the non-cancerous cell line). FIG. 4D shows images demonstrating presence of the FITC-conjugated myr-NLS peptide (myr-NLS-FITC, SEQ ID NO: 6) inside MCF7 cells following 1 hour of treatment with 7.5 μM peptide (nuclei are stained with Hoechst). FIG. 4E shows comparison of the effects of myr-NLS (SEQ ID NO: 5) to myr-NLS-FITC, (SEQ ID NO: 6) on cell viability in MCF7 breast cancer cells (treatment was induced with M peptide for 72 hours); p-value <0.005; *—compared to untreated; \hat{ }—compared to vehicle treated (untreated).

FIGS. 5A-C demonstrate the effect of the synthesized stapled myr-NLS peptides on viability of cancerous cells. FIG. 5A is a bar graph demonstrating the effect of myr-NLS (SEQ ID NO: 5) and several stapled variants of the peptide (SEQ ID Nos: 7-11) on viability of SKBR3 and MCF7 cells (treatment was induced with 30 μM peptide for 48 hours; p-value <0.005; *—compared to untreated; \hat{ }—compared to myr-NLS (SEQ ID NO: 3)). FIG. 5B demonstrate the effect of mN6-10 (SEQ ID NO: 7) on viability of cancerous cell lines (MCF7 left panel, breast; Rat-1-EJ right panel, fibroblasts transformed with constitutively active Ras) as compared to their respective non-cancerous cells (MCF10A; Rat-1), and their respective IC-50, as determined for each cell line. (p-value <0.005; *—regression analysis; \hat{ }—compared to the non-cancerous cell line). FIG. 5C shows dose-response analysis of the effect of mN6-10 peptide (SEQ ID NO: 7) on viability of glioma (U87), breast (SKBR3), pancreatic (Panc-1) and colon (HCT-116 and DLD-1) cancer cells, following treatment for 72 hours. IC-50 value for each cell line was determined (p-value <0.005; *—regression analysis).

FIGS. 6A-B demonstrate the effect of the synthesized stapled myr-NLS peptides (SEQ ID Nos: 7-11) on viability of cells. FIG. 6A shows bar graphs demonstrating the effect of the indicated peptides on viability of cancerous cell lines (MCF7 left panel, breast; EJ right panel, fibroblasts transformed with constitutively active Ras) as compared to their respective non-cancerous cells (MCF10A; Rat-1) following 72 hours of treatment with 10 μM peptide in the breast cells lines and 20 μM in the fibroblasts cell lines (p-value <0.01; *—compared to untreated; #—compared to myr-NLS (SEQ ID NO: 3)). FIG. 6B demonstrate dose response analysis of the effect of the mN8-12 peptide (SEQ ID NO: 9) on viability of MCF10A and MCF7 cells (left), and Rat-1 and EJ cells (right), and their respective IC-50, as determined for each cell line (p-value <0.005; *—regression analysis).

FIGS. 7A-C demonstrate the effect of combined treatment with a TAT-NLS peptide (SEQ ID NO: 3) and GroA (AS1411). FIG. 7A demonstrates viability assessment of SKBR3 cells treated for 72 hours with increased concentrations of GroA (AS1411), TAT-NLS (SEQ ID NO: 3) or a combination of both, as indicated (p-value <0.05; *—regression analysis; \hat{ }—compared to monotherapy with GroA or TAT-NLS). FIG. 7B is a bar graph demonstrating viability of MCF10A and MCF7 cells treated for 72 hours with GroA (AS1411, 5 μM), TAT-NLS (SEQ ID NO: 3, 15 μM) or a combination of both, as indicated (p-value <0.05; *—compared to control; #—compared to GroA or myr-NLS alone; \hat{ }—MCF7 compared to MCF10A, as indicated). FIG. 7C demonstrate dose response analysis of the effect of GroA or myr-NLS on viability of MCF7 and Panc-1 cells, following 72 hours of treatment (p-value <0.005; *—regression analysis; \hat{ }-treatments comparison).

FIG. 8 shows the anti-tumor effect of the myr-NLS peptide (SEQ ID NO: 5) as determined in a colony formation assay. Colony formation was tested in Panc-1 or MIA PaCa-2 cells, following 72 hours treatment with myr-NLS1 (SEQ ID NO: 5) (8 μM and 4.5 μM for Panc-1 and MIA PaCa-2, respectively). Representative images are presented in the left panel. Quantification of the results is presented on the right panel and is the fold induction compared to the untreated plates. (mean±SE; n=10; *,*** (p-value<0.05, 0.005, respectively).

FIGS. 9A-C demonstrate that the myr-NLS peptide (SEQ ID NO: 5) inhibits pancreatic tumor growth in-vivo. Nude mice bearing Panc-1 (pancreatic cancer) tumor xenografts were treated daily with 25 μg myr-NLS peptide (SEQ ID NO: 5, marked as myr-JM) or vehicle control. FIG. 9A is a graph demonstrating tumor size. Shown is Mean±SE; n>6; ***, \hat{ }\hat{ }\hat{ }—p-value <0.005, regression and slopes and intercepts difference analysis, respectively. FIG. 9B is a graph demonstrating tumor weight. Shown is mean±SE; n>5; *—p-value <0.05. FIG. 9C shows representative images of the treated mice.

FIGS. 10A-C demonstrate the in-vivo effect of the myr-NLS peptide (SEQ ID NO: 5) on protein expression in the mouse Panc-1 tumor xenografts model described in FIGS. 9A-C. FIG. 10A is a western blot image demonstrating the levels of the indicated proteins in Panc-1 tumors dissected from vehicle control mice compared to myr-NLS peptide (SEQ ID NO: 5)—treated mice. Proteins and phosphorylated proteins (p) levels of the ErbB/HER receptors (EGFR (ErbB1) and ErbB2), Erk, Akt and Nucleolin were determined by western blot using the relevant antibodies, as indicated. FIGS. 10B-C are graphs demonstrating quantification of the protein levels image. Quantitation was performed using ImageJ program and normalized to actin protein level (FIG. 10B) or to total protein expression level (FIG. 10C). The values are fold inducted to the mean vehicle values. Shown is mean±SE; n>5; *, **—p-value<0.05 and 0.01, respectively.

FIGS. 11A-C demonstrate dose-response effect of the myr-NLS1 (SEQ ID NO: 5), myr-NLS2 (SEQ ID NO: 15), myr-NLS3 (SEQ ID NO: 16) and myr-NLS4 (SEQ ID NO: 17) peptides on viability of pancreatic (Panc-1, MIA PaCa-2) and colon (DLD-1) cancer cells, following treatment for 72 hours. Inhibitory concentration (IC-50) was determined for each cell line (p-value <0.005; ***—regression analysis).

FIG. 12 demonstrates the anti-tumor effect of myr-NLS1 (SEQ ID NO: 5), myr-NLS2 (SEQ ID NO: 15) and myr-NLS4 (SEQ ID NO: 17) peptides, as determined in a colony formation assay. Colony formation was tested in DLD-1 and HCT116 colon cancer cells treated or untreated for 72 hours with the indicated peptides (20 μM and 13 μM, respectively). Representative images are presented in the left panels. Quantification of the results are presented in the right panels and are presented as the fold induction compared to the untreated plates. (mean±SE; n=10; *** ; p-value<0.005).

FIGS. 13A-B demonstrate the levels of ErbB2, pErbB2, EGFR, pEGFR, nucleolin, pAkt, Akt, pErk and Erk following treatment with a myr-NLS1 (SEQ ID NO: 5) peptide in DLD-1 (FIG. 13A) and Panc-1 (FIG. 13B) cell lines, as determined by western-blot analysis. In each Figure: Upper panel shows representative blots; and lower panel shows quantification analyses. (25 μM and 10 μM for DLD-1 and Panc-1, respectively, 24 hours; * p-value <0.05, ** p-value <0.01, *** p-value <0.005 compared to untreated cells).

FIGS. 14A-C demonstrate the in-vivo effect of myr-NLS1 (SEQ ID NO: 5) and Myr NLS4 (SEQ ID NO: 17) peptide, in a mouse model of colon cancer xenograft. Nude mice bearing DLD-1 colon cancer tumor xenografts were daily treated with: vehicle, myr-NLS and myr-NLS4 (25 μg/100 μl PBS). FIG. 14A is a graph showing the mean tumor size (mean±SE; ***, \hat{ }\hat{ }\hat{ } p-value <0.005). FIG. 14B) is a graph showing mean tumor weight (mean±SE; n>8; * p-value <0.05 compared to vehicle control). FIG. 14C shows representative images of the treated mice.

### DESCRIPTION OF SPECIFIC EMBODIMENTS OF THE INVENTION

The present invention, in some embodiments thereof, relates to HER receptor derived peptides and methods of use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The HER family proteins have been described in involvement of several pathologies, including psoriasis, atherosclerosis, and several types of human cancer. Consistent with their essential roles in tumor progression, strategies able to interfere with HER functions, such as monoclonal antibodies (mAbs) and tyrosine kinase inhibitors (TKIs), have yielded in the last several decades several oncology drugs. However, many cancers are resistant to HER targeted therapy and many others become resistant following prolonged treatment.

Nucleolin is a multifunctional protein frequently deregulated in cancer cells. This protein was shown to directly interact with HER receptors and with Ras; and this interaction has been shown to lead to ligand-independent activation of the receptor and to increased cell transformation in in EGFR, HER2 and Ras-driven cancers [6-13].

Whilst reducing specific embodiments of the present invention to practice, the present inventors have generated peptides comprising the nuclear localization sequence (NLS) of HER receptors (EGFR, HER2, HER3 or HER4), which is the interacting domain of the HER receptor with nucleolin. Further, various stapled peptides based on the NLS sequences were also generated. All these peptides were modified to increase peptide penetration into cells by adding a TAT sequence, a poly R sequence or myristoyl (Examples 1-2 and 5 of the Examples section which follows). These modified peptides reduced the interaction between the HER receptors and nucleolin, reduced expression and activation of HER receptors, and inhibited growth of several types of tumor cells (e.g. colon, pancreas, breast) both in-vitro and in in-vivo, while not affecting viability of non-cancerous cells (Examples 1-2 and 4-5 of the Examples section which follows). Importantly, unmodified peptides had no significant activity (Example 1 of the Examples section which follows). Further, combined treatment with the modified peptides and a known anti-nucleolin G-rich oligonucleotide, GroA, was significantly more effective compared to treatment with each of them alone (Example 3 of the Examples section which follows).

Thus, according to an aspect of the present invention, there is provided a composition of matter comprising a peptide comprising an amino acid sequence of a nuclear localization sequence (NLS) of a HER receptor capable of binding a nucleolin polypeptide, said peptide being attached to a cell penetrating moiety.

As used herein, the terms “HER receptor” and “ErbB receptor”, which are interchangeably used herein, refer to receptors of the ErbB family of receptor tyrosine kinases E.C. 2.7.10.1 including EGFR, HER2, HER3 and HER4.

According to specific embodiments, the HER receptor is EGFR.

As used herein “EGFR” refers to a receptor tyrosine kinase (RTK) of the epidermal growth factor receptor family, also referred to as HER1, mENA and ErbB-1. According to a specific embodiment the EGFR is human EGFR i.e., EGFR_HUMAN, P00533.

According to specific embodiments, the HER receptor is selected from the group consisting of HER2, HER3 and HER4.

According to specific embodiments, the HER receptor is HER2.

As used herein “HER2” refers to a receptor tyrosine kinase (RTK) of the epidermal growth factor receptor family, also referred to as ErbB-2, NEU and p185erbB-2. According to a specific embodiment the HER2 is human HER2 i.e., ERBB2_HUMAN, P04626.

According to specific embodiments, the HER receptor is HER3.

As used herein “HER3” refers to a receptor tyrosine kinase (RTK) of the epidermal growth factor receptor family, also referred to as ErbB-3. According to specific embodiments the HER3 is human HER3 i.e., ERBB3_HUMAN, P21860.

According to specific embodiments, the HER receptor is HER4.

As used herein “HER4” refers to a receptor tyrosine kinase (RTK) of the epidermal growth factor receptor family, also referred to as ErbB-4. According to specific embodiments the HER4 is human HER4 i.e., ERBB4_HUMAN, Q15303.

As used herein the phrase “nuclear localization sequence (NLS) of a HER receptor” refers to at least a fragment, a non-consecutive sequence and/or a homolog of an NLS sequence of a HER receptor (also known as ErbB).

According to specific embodiments, the amino acid sequence of the NLS does not exceed 25, amino acids, 20 amino acids, 16 amino acids or 15 amino acids.

According to a specific embodiment, the amino acid sequence of the NLS does not exceed 16 amino acids.

According to a specific embodiment, the amino acid sequence of the NLS does not exceed 15 amino acids.

According to specific embodiments, the amino acid sequence of the NLS is at least 8, at least 9, at least 10, at least 11, at least 12, at least 13, at least 14 or at least 15 amino acids long.

According to a specific embodiment, the amino acid sequence of the NLS is at least 10 amino acids long.

According to a specific embodiment, the amino acid sequence of the NLS is 10-20, 10-16, 12-16, 13-16, 14-16 or 15-16 amino acids long.

According to a specific embodiment, the amino acid sequence of the NLS is 15-16 amino acids long.

According to a specific embodiment, the amino acid sequence of the NLS is capable of binding a nucleolin polypeptide.

Alternatively or additionally, according to a specific embodiment, the amino acid sequence of the NLS is capable of inhibiting binding of the HER receptor to a nucleolin polypeptide.

Alternatively or additionally, according to a specific embodiment, the amino acid sequence of the NLS is capable of inhibiting activation of the HER receptor upon binding to a HER ligand. HER ligands are known in the art and include for example EGF, TGFα, betacellulin, amphiregulin, HB-EGF, epiregulin and neuregulins.

Nuclear localization sequences of HER receptors are known in the art, such as, but not limited to, RRRHIVRKRTLRRLL (SEQ ID NO: 1)—the NLS sequence of human EGFR (also known as HER1 or ErbB1), KRRQQKIRKYTMRRLL (SEQ ID NO: 12)—the NLS sequence of human HER2 (also known as ErbB2), RGRRIQNKRAMRRYL (SEQ ID NO: 13)—the NLS sequence of human HER3 (also known as ErbB3), RRKSIKKKRALRRFL (SEQ ID NO: 14)—the NLS sequence of human HER4 (also known as ErbB4).

According to specific embodiments, the homolog is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to an NLS sequence of a HER receptor such as provided e.g. in SEQ ID Nos: 1, 12, 13 or 14.

Sequence identity can be determined using any protein sequence alignment algorithm such as Blast and ClustalW.

According to specific embodiments, the peptide comprises SEQ ID NO: 1.

According to specific embodiments, the peptide consists of SEQ ID NO: 1.

According to specific embodiments, the homolog comprises at least one amino acid substitution at an amino acid position selected form the group consisting of V6, T10, R7, L11, K8, R12, L11 and L15 of SEQ ID NO: 1.

According to specific embodiments, the homolog comprises at least one amino acid substitution at an amino acid position selected form the group consisting of V6, T10, L11 and L15 of SEQ ID NO: 1.

According to a specific embodiment, the substitution is a conservative substitution as further described hereinbelow.

According to a specific embodiment, the substitution is a non-conservative substitution as further described hereinbelow.

According to specific embodiments, the peptide comprises SEQ ID NO: 12, 13 or 14.

According to specific embodiments, the peptide consists of SEQ ID NO: 12, 13 or 14.

According to specific embodiments, the peptide comprises SEQ ID NO: 12.

According to specific embodiments, the peptide consists of SEQ ID NO: 12.

According to specific embodiments, the peptide comprises SEQ ID NO: 13.

According to specific embodiments, the peptide consists of SEQ ID NO: 13.

According to specific embodiments, the peptide comprises SEQ ID NO: 14.

According to specific embodiments, the peptide consists of SEQ ID NO: 14. As used herein, the term “nucleolin”, refers to the polypeptide expression product of the NCL gene (corresponding to the human Gene ID 4691). According to specific embodiments, nucleolin is human nucleolin, such as provided in the following GenBank Accession No. NP_005372.

Assays for testing binding are well known in the art and include, but not limited to flow cytometry, ELISA, immunoprecipitation, BiaCore, bio-layer interferometry Blitz® assay, HPLC, surface plasmon resonance. Alternatively or additionally, binding can be assessed by determining activity, e.g. inhibition of activation of a HER receptor by phosphorylation of the downstream signaling cascade, including MAPK/ERK and PI(3)K/Akt using e.g. western blow, immunoprecipitation, phosphospecific antibodies, reporter gene assays etc.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)—CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2—CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino | | | |
|---|---|---|---|
| acid | Code | Non-conventional amino acid | Code |
| ornithine | Orn | hydroxyproline | Hyp |
| α-aminobutyric acid | Abu | aminonorborny1- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl) | Nnbhm |
| diphenylethyl)glycine | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4- | Tic |
| ethylamino)cyclopropane | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The amino acids of the polypeptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid. Those non-conservative substitutions which fall under the scope of the present invention are those which still constitute a peptide having neuroprotective properties.

The peptides of some embodiments of the invention are utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

According to specific embodiments, the peptide is a stapled peptide.

As used herein, the term “stapled peptide” refers to a peptide comprising at least one pair of functionalized amino acids, wherein the functionalized amino acids are joined by a staple. The stapled peptide may comprise a single or multiple staples. Multiply stapled peptide refers to a peptide containing more than one individual staple, and may contain two, three, or more independent staples of various spacings and compositions. Peptides staples and methods of producing a stapled peptide are known in the art and include, but are not limited to, hydrocarbon cross-links and triazole-containing (e.g, 1, 4 triazole or 1, 5 triazole) crosslinks, and disclosed e.g. in M. Pelay-Gimeno, et al. (2015), Angew. Chem. Int. Ed. 54, 8896-8927; Walensky, LD; Bird, GH (2014), Journal of Medicinal Chemistry, 57 (15): 6275-88; Schafmiester et al. (2000), J. Am. Chem. Soc, 122:5891-5892; Walensky et al. (2004), Science, 305: 1466-1470; Kawamoto et al. (2012), Journal of Medicinal Chemistry 55: 1137; and International Patent Application Publication No. WO 2010/060112, the contents of which are fully incorporated herein by reference.

According to specific embodiments, the peptide stapling is effected by covalently joining two olefin-containing side-chains present in the peptide using a ring-closing metathesis (RCM) reaction to form a cross-linked ring (see e.g. Blackwell, Helen E.; Grubbs, Robert H. (1998). Angewandte Chemie International Edition. 37 (23): 3281-3284; Blackwell et al, J. (2001) Org. Chem., 66: 5291-5302; and Angew et al. (1994) Chem. Int. Ed. 37:3281, the contents of which are fully incorporated herein by reference).

According to specific embodiments, stapling is effected by replacing peptide amino acids residues i and i+4 with S-pentenylalanine (S5); by replacing peptide amino acids residues i and i+7 with R-octenylalanine (R8) and S-pentenylalanine, respectively; or by replacing peptide amino acids residues i and i+7 with S-octenylalanine and R-pentenylalanine, respectively.

Non-limiting examples of stapled peptides comprising an amino acid sequence of a NLS of a HER receptor which can be used with specific embodiments of the present invention include SEQ ID Nos: 18-22 and 7-11.

According to specific embodiments, the stapled peptide comprises SEQ ID NO 18, 19, 20, 21 or 22.

According to specific embodiments, the stapled peptide consists of SEQ ID NO: 18, 19, 20, 21 or 22.

According to specific embodiments, the stapled peptide comprises SEQ ID NO 18 or 21.

According to specific embodiments, the stapled peptide consists of SEQ ID NO: 18 or 21.

According to specific embodiments, the stapled peptide comprises SEQ ID NO: 7 or 10.

According to specific embodiments, peptide stapling increases peptide's resistance to proteolytic cleavage, increases peptide's thermal stability, increases peptide's hydrophobicity, allows for better penetration of the peptide into cells and/or improves peptide's biological activity relative to the corresponding unstapled peptide.

The peptide of some embodiments of the invention is attached to a cell penetrating moiety.

According to specific embodiments, the peptide is attached a cell penetrating moiety.

As used herein the phrase “cell penetrating moiety” refers to a moiety which enhances translocation of the peptide across a cell membrane. Non-limiting examples of cell penetrating moieties include, but not limited to, a myristoyl group, a lipid a nanoparticle, a liposome, a cell penetrating peptide and poly(alkylene) glycols.

“Attached to”, as used herein in this context, includes both covalent or non-covalent attachment of the cell penetrating moiety to the peptide and encapsulation of the peptide by the cell penetrating moiety.

For example, the peptide may be incorporated into a particulated delivery vehicle, e.g., a liposome, or a nano- or microparticle, by any of the methods known in the art [e.g. Liposome Technology, Vol. II, Incorporation of Drugs, Proteins, and Genetic Material, CRC Press; Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43; Ramishetti et al. Adv Mater. 2020 Jan. 30:e1906128, International Patent Application publication Nos. WO2018/015881 and WO2018087753, WO2017194454 and US Patent Application Publication no. US20130245107, the contents of which are fully incorporated herein by reference].

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. Liposomes can be of different sizes, may contain a low or a high pH and may be of different charge.

Method of binding a particle to a peptide are known in the art and are also disclosed in e.g. International Patent Application Publication NO. WO2018/015881, U.S. Pat. Nos. 5,171,578, 5,204,096 and 5,258,499, the contents of which are fully incorporated herein by reference.

According to specific embodiment, the cell penetrating moiety is a fatty acid moiety. Examples of fatty acids that may be used as a cell penetrating component according to the present invention include caprylic acid (octanoic acid; C8:0), capric acid (decanoic acid; C10:0), lauric acid (dodecanoic acid; C12:0), myristic acid (tetradecanoic acid; C14:0), palmitic acid (hexadecanoic acid), C16:0, stearic acid (octadecanoic acid), C18:0), arachidic acid (eicosanoic acid, C20:0), behenic acid (docosadecanoic acid), C22:0), lignoceric acid (tetracosanoic acid), C24:0, cerotic acid (hexacosanoic acid). Such a fatty acid moiety may be covalently linked to the peptide backbone, for example by acylation, for example by N-myristoylation or palmitoylation.

According to specific embodiments, the cell penetrating moiety is a myristoyl group.

The term “myristoyl group” or “myristoylation” means that myristoyl is conjugated to the amino acid, in particular the alpha-amino group of the N-terminal residue, of the peptide of the invention via an amide bond.

According to specific embodiments, the cell penetrating moiety is a cell penetrating peptide.

Hence, according to specific embodiments, the peptide is attached to the cell penetrating moiety via a peptide bond.

As used herein, a “cell-penetrating peptide (CPP)” is a peptide that comprises a short (about 12-40 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention may include, but are not limited to, TAT [e.g. TAT(48-60)], poly R sequence, penetratin, transportan, pIs1, pVEC, MTS, and MAP. According to specific embodiments, the cell penetrating peptide is selected from the group consisting of TAT and poly R sequence peptides Protocols for producing CPPs-cargos conjugates can be found, for example L Theodore et al. [The Journal of Neuroscience, (1995) 15(11): 7158-7167], Fawell S, et al. [Proc Natl Acad Sci USA, (1994) 91:664-668], and Jing Bian et al. [Circulation Research. (2007) 100: 1626-1633].

Non-limiting Examples of compositions that can be used with specific embodiments of the invention are described in the Examples section which follows and include SEQ ID Nos: 3-11 and 15-17.

The peptides and compositions comprising same of some embodiments of the invention may be synthesized by any techniques that are known to those skilled in the art of peptide synthesis, including both synthetic methods and recombinant techniques.

According to an aspect of the present invention, there is provided a method of producing a peptide, the method comprising chemically synthesizing the composition of matter disclosed herein.

According to an additional aspect of the present invention, there is provided a method of producing a peptide, the method comprising chemically synthesizing a peptide comprising an amino acid sequence of a NLS of a HER receptor capable of binding a nucleolin polypeptide, said amino acid sequence of said NLS is 16 amino acids long or less.

Non-limiting examples of chemical methods of peptide synthesis include solid phase peptide synthesis, liquid phase synthesis, chemical ligation, and chemical modifications (e.g. conjugation, acylation, alkylation) techniques.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

According to specific embodiments, the peptide is synthesized using in vitro expression systems. Such in vitro synthesis methods are well known in the art and the components of the system are commercially available.

According to specific embodiments, the peptide is produced by recombinant DNA technology. A “recombinant” peptide, or protein refers to a peptide, or protein produced by recombinant DNA techniques; i.e., produced from cells transformed by an exogenous DNA construct encoding the desired peptide or protein.

Thus, according to another aspect of the present invention, there is provided an isolated polynucleotide comprising a nucleic acid sequence encoding any of the above described compositions.

According to an aspect of the present invention, there is provided an isolated polynucleotide encoding a peptide comprising an amino acid sequence of a NLS of a HER receptor capable of binding a nucleolin polypeptide, said amino acid sequence of said NLS does not exceed 16 amino acids.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

To express an exogenous polypeptide in mammalian cells, a polynucleotide sequence encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

Hence, according to specific embodiments, there is provided nucleic acid construct comprising the polynucleotide and a regulatory element for directing expression of the polynucleotide in a host cell.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

According to specific embodiments, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the peptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of some the compositions disclosed herein as they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods. Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

As mentioned, other than containing the necessary elements for the transcription and translation of the inserted coding sequence, the expression construct of some embodiments of the invention can also include sequences engineered to enhance stability, production, purification, yield or toxicity of the expressed peptide. For example, the expression of a fusion protein or a cleavable fusion protein comprising the disclosed peptide and a heterologous protein can be engineered. Such a fusion protein can be designed so that the fusion protein can be readily isolated by affinity chromatography; e.g., by immobilization on a column specific for the heterologous protein. Where a cleavage site is engineered, the peptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

The present invention also contemplates cells comprising the composition described herein.

Thus, according to an aspect of the present invention, there is provided a host cell comprising the composition of matter, the polynucleotide or the nucleic acid construct disclosed herein.

As mentioned hereinabove, a variety of prokaryotic or eukaryotic cells can be used as host-expression systems to express the peptide of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the peptides of some embodiments of the invention.

Examples of bacterial constructs include the pET series of E. coli expression vectors (Studier et al. (1990) Methods in Enzymol. 185:60-89).

Examples of eukaryotic cells which may be used along with the teachings of the invention include but are not limited to, mammalian cells, fungal cells, yeast cells, insect cells, algal cells or plant cells.

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art can also be used by some embodiments of the invention.

According to specific embodiments the cell is a mammalian cell.

According to specific embodiment, the cell is a human cell.

According to a specific embodiment, the cell is a cell line.

According to another specific embodiment, the cell is a primary cell.

The cell may be derived from a suitable tissue including but not limited to blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells may be derived from any developmental stage including embryo, fetal and adult stages, as well as developmental origin i.e., ectodermal, mesodermal, and endodermal origin.

According to specific embodiments, the cell is not derived from an embryo.

Non limiting examples of mammalian cells include monkey kidney CV1 line transformed by SV40 (COS, e.g. COS-7, ATCC CRL 1651); human embryonic kidney line (HEK293 or HEK293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HeLa, ATCC CCL 2); NIH3T3, Jurkat, canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2), PER.C6, K562, and Chinese hamster ovary cells (CHO).

Consequently, according to an aspect of the present invention, there is provided a method of producing a peptide, the method comprising introducing into a host cell the polynucleotide or the nucleic acid construct described herein or culturing the host cell described herein.

According to specific embodiments, the methods comprising isolating the peptide.

According to specific embodiments, recovery of the recombinant peptide is effected following an appropriate time in culture. The phrase “recovering the recombinant peptide” refers to collecting the whole fermentation medium containing the peptide and need not imply additional steps of separation or purification. Notwithstanding the above, peptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, mix mode chromatography, metal affinity chromatography, Lectins affinity chromatography, chromatofocusing and differential solubilization.

According to specific embodiments, following synthesis and/or purification, the activity (e.g. therapeutic efficacy) of the peptide can be assayed either in vivo or in vitro. Such methods are known in the art and are also described in the Example section which follows and include for example, but not limited to, binding assays, cell penetration assays, cell viability, therapeutic effect and/or survival of animal models (e.g. mice) and the like. The present teachings also contemplate the use of the compositions disclosed herein in methods of treating a disease associated with a HER receptor.

Thus, according to an aspect of the present invention, there is provided a method of treating a disease associated with a HER receptor, the method comprising administering to the subject a therapeutically effective amount of the composition of matter, the polynucleotide, the nucleic acid construct, or the host cell described herein, thereby treating the disease in the subject.

According to an additional or an alternative aspect of the present invention, there is provided the composition of matter, the polynucleotide, the nucleic acid construct, or the host cell described herein, for use in treating a disease associated with a HER receptor.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender. According to specific embodiments, the term “subject” refers to a subject who suffers from the pathology (disease, disorder or medical condition). According to specific embodiments, this term encompasses individuals who are at risk to develop the pathology.

As used herein the phrase, “disease associated with a HER receptor” includes diseases in which overexpression and/or unregulated activation of a HER receptor drives onset and/or progression of the disease. Such overexpression or unregulated activation can be a result of amplification and/or mutation of a HER receptor and is usually manifested by increased level of the receptor on the cell surface of pathologic cells and/or over-activation of the receptor (e.g. constitutive activity which is typically independent of ligand binding).

According to specific embodiments, pathologic cells of the disease overexpress the HER receptor associated with the disease as compared to non-pathologic cells of the same origin.

According to specific embodiments, pathologic cells of the disease overexpress nucleolin as compared to non-pathologic cells of the same origin.

According to specific embodiments, pathologic cells of the disease overexpress Ras as compared to non-pathologic cells of the same origin.

Ras, as used herein, includes both wild-type and oncogenic (i.e. constitutively active) Ras. Examples of Ras include H-Ras, N-Ras, K-ras.

As used herein, “overexpress” or “overexpression” refers to higher levels of a gene product (e.g. mRNA, polypeptide) in cells associated with the disease as compared to non-pathologic cells of the same origin. According to specific embodiments, overexpression result in an increased level of at least 2%, at least 5%, at least 10%, at least 20%, at least 30%, at least 40%, at least 50%, at least 100% or more, as compared to the non-pathologic cell. Methods of determining overexpression are well known in the art and include, but not limited to PCR, western blot, flow cytometry and immuno-cytochemistry. Specific methods suitable for determining cell surface presentation include, but not limited to flow cytometry and immuno-cytochemistry. Alternatively or additionally, overexpression can be determined by assessing activation of the receptor by phosphorylation of the downstream signaling cascade, including MAPK/ERK and PI(3)K/Akt using e.g. western blow, immunoprecipitation, phosphospecific antibodies, reporter gene assays etc.

Non-limiting examples diseases that can be treated according to some embodiments of the invention include cancer, psoriasis and atherosclerosis.

According to specific embodiments, the disease is cancer.

Cancers which may be treated by some embodiments of the invention can be any solid or non-solid tumor, cancer metastasis and/or a pre-cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Pre-cancers are well characterized and known in the art (refer, for example, to Berman J J. and Henson D E., 2003. Classifying the precancers: a metadata approach. BMC Med Inform Decis Mak. 3:8). Examples of precancers include but are not limited to include acquired small precancers, acquired large lesions with nuclear atypia, precursor lesions occurring with inherited hyperplastic syndromes that progress to cancer, and acquired diffuse hyperplasias and diffuse metaplasias. Non-limiting examples of small precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

According to specific embodiments, the cancer is selected from the group consisting of breast cancer, prostate cancer, pancreatic cancer, ovarian cancer, colon cancer and glioma.

According to specific embodiments, the cancer is colon cancer (e.g. colon carcinoma).

According to specific embodiments, the cancer is pancreatic cancer (e.g. pancreatic carcinoma e.g. of ductal cell origin).

According to specific embodiments, the cancer is breast cancer.

According to specific embodiments, the cancer is glioma.

According to specific embodiments, the compositions disclosed herein can be administered to a subject in combination with other established or experimental therapeutic regimen to treat a disease associated with a HER receptor (e.g. cancer) including, but not limited to analgesics, small molecules, chemotherapeutic agents, radiotherapeutic agents, cytotoxic therapies, hormonal therapy, adoptive cell transplantation (e.g. tansplantation of bone marrow cells, hematopoietic stem cells, PBMCs, cord blood stem cells and/or induced pluripotent stem cells), antibodies and other treatment regimens (e.g., surgery) which are well known in the art.

According to specific embodiments, the therapeutic agent administered in combination with the composition of some embodiments of the invention comprises an anti-cancer agent.

Anti-cancer agents that can be use with specific embodiments of the invention include, but are not limited to the anti-cancer drugs Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

According to specific embodiments, the anti-cancer agent comprises an antibody.

According to specific embodiments, the antibody is selected from the group consisting rituximab, cetuximab, trastuzumab, edrecolomab, alemtuzumab, gemtuzumab, ibritumomab, panitumumab, Belimumab, Bevacizumab, Bivatuzumab mertansine, Blinatumomab, Blontuvetmab, Brentuximab vedotin, Catumaxomab, Cixutumumab, Daclizumab, Adalimumab, Bezlotoxumab, Certolizumab pegol, Citatuzumab bogatox, Daratumumab, Dinutuximab, Elotuzumab, Ertumaxomab, Etaracizumab, Gemtuzumab ozogamicin, Girentuximab, Necitumumab, Obinutuzumab, Ofatumumab, Pertuzumab, Ramucirumab, Siltuximab, Tositumomab, Trastuzumab, Nivolumab, Pembrolizumab, Durvalumab, Atezolizumab, Avelumab and ipilimumab.

According to specific embodiments, the additional therapeutic agent is specific for a HER receptor, nucleolin and/or Ras.

As used herein, the phrase “specific for” refers to a therapeutic agent having a moiety (e.g. an antibody binding domain, a ligand, a receptor, a leptin etc.) having a binding affinity (e.g. below 10⁻⁴ nM) to the recited target. Assays for testing binding are well known in the art and are further described herein. According to specific embodiments, specificity is mediated by CDR's being specific to a target antigen.

Therapeutic agents specific for a HER receptor, nucleolin and/or Ras are well known in the art.

Thus, for example a therapeutic agent may be an anti-HER receptor antibody, an anti-nucleolin antibody and/or an anti-Ras antibody.

Non-limiting examples of anti-HER receptor antibodies that can be used with specific embodiments of the invention include trastuzumab (Herceptin), Pertuzumab, cetuximab (Erbitux), panitumumab (Vectibix), and seribantumab.

Another non-limiting example for a therapeutic agents may be a small molecule.

Non-limiting examples small molecules specific for a HER receptor that can be used with specific embodiments of the invention include tyrosine kinase inhibitors (TKIs) such as, but not limited to erlotinib HCL (OSI-774; Tarceva®; OSI Pharma), gefitinib (Iressa®, AstraZeneca and Teva), lapatinib (Tykerb®, GlaxoSmithKline), canertinib (CI-1033, PD183805; Pfizer), PKI-166 (Novartis); PD158780; pelitinib; AG 1478 (4-(3-Chloroanillino)-6,7-dimethoxyquinazoline), canertinib (CJ-1033, PD 183805; Pfizer) and Zactima (ZD6474), perlitinib (EKB-569), neratinib (HKI-272), tucatinib (Tukysa), vandetanib (ZD6474), afatinib, dacomitinib, AZD9291, rociletinib (CO-1686), HM61713 and WZ4002.

Non-limiting examples agents specific for Ras that can be used with specific embodiments of the invention include Kras G12C inhibitors [such as sotorasib (AMG510)], MEK inhibitors (such as trametinib and cobimetinib) and PI3K inhibitors (such as idelalisib).

Another non-limiting example for a therapeutic agents may be an aptamer.

A non-limiting example of an aptamer specific for nucleolin that can be used with specific embodiments of the invention is GroA (AS1411 or AGRO100, can be commercially obtained from e.g. IDT (Jerusalem, Israel).

According to specific embodiments the combination therapy has an additive effect.

According to specific embodiments, the combination therapy has a synergistic effect.

The composition of some embodiments of the invention can be administered to a subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the composition (e.g. a composition comprising a peptide comprising an amino acid sequence of a NLS of a HER receptor being attached to a cell penetrating moiety, polynucleotide or nucleic acid construct encoding same or host cell comprising same) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-IT Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Materials and buffers—The following antibodies were used: monoclonal mouse anti-actin (691001; MP Biomedicals, Santa Ana, CA); polyclonal rabbit anti-ErbB2 (HER2/neu, sc-284; Santa Cruz Biotechnology, Dallas, TX); rabbit anti-EGF (sc-03; Santa Cruz Biotechnology); monoclonal mouse anti-nucleolin (sc-8031; Santa Cruz Biotechnology); polyclonal rabbit anti-phospho-ErbB2 (2249; Cell Signaling Technology, Danvers, MA); and polyclonal rabbit anti-phospho-EGFR (SAB4300063; Sigma-Aldrich). Secondary antibodies used were polyclonal donkey anti-mouse-HRP and goat anti-rabbit-HRP (715035151 and 111035144, respectively; Jackson ImmunoResearch). The aptamer GroA (AS1411) was purchased from IDT (Jerusalem, Israel) as unmodified desalted oligonucleotides, as previously described [4, 15]. Solubilization buffer contained 50 mM HEPES (pH 7.5), 150 mM NaCl, 1% Triton X-100, 1 mM EGTA, 1 mM EDTA, 1.5 mM MgCl2, 10% glycerol, 200 μM sodium vanadate, and 1 μmol/L inhibitors cocktail (EMD Millipore, 539131).

Cell lines—Human breast cancer cell lines SKBR3 [Peles E et al. EMBO J. (1993) 12(3):961-71] and MCF7 [Faigenbloom L et al., Mol Syst Biol. (2015) 11(12):845] and human glioblastoma U87 [Goldberg L et al. Cancer Res. (2006) 66(24):11709-17] cell line, as well as Rat-1 fibroblast cells and H-Ras-transformed Rat-1 cells (EJ cells) [Marom M et al. J Biol Chem. (1995) 270(38):22263-70] and human Panc-1 and MIA PaCa-2 pancreatic cell lines (Rak R et al. Oncoscience. 2014 1(1):39-48; Weisz B et al. Oncogene (1999) 18, 2579±2588) were all grown in Dulbecco's modified Eagle's medium (DMEM; Biological Industries, Beithaemek, Israel). Human colorectal cancer cell lines DLD-1 (ATTC Cat No. CCL-221), HCT-116 (ATTC Cat No. CCL-247) and LNCaP prostate cancer cells were grown in Roswell Park Memorial Institute 1640 (RPMI 1640) medium and McCoy's 5A media, respectively (Sigma-Aldrich). All media were supplemented with antibiotics and 10% heat-inactivated fetal bovine serum (FBS; Gibco). Human breast cell line MCF10A [Bott A et al. Oncotarget. (2017) 8(27):43897-43914] was grown in Dulbeco's Modified Eagle's Medium/Nutrient Mixture F-12 HAM medium (DMEM F-12 HAM; Sigma-Aldrich), supplemented with antibiotics, 5% inactivated horse serum (HS; Invitrogen), 2 mM L-glutamine (Biological Industries, Beithaemek, Israel), 0.1 μg/ml cholera toxin (Sigma-Aldrich), 0.5 μg/ml hydrocortisone (Sigma-Aldrich), 10 μg/ml insulin (Biological Industries, Beithaemek, Israel) and 2 ng/ml EGF (R&D Systems). Cells were incubated at 37° C. in 5% CO₂ in air, and the medium was changed every 3-4 days. Cells were passaged in trypsin/disodium ethylenediaminetetraacetic acid (Biological Industries, Beithaemek, Israel) when confluency reached 70%. One day prior to treatment the cells were plated at ~50% confluency in a medium supplemented with 10% FBS or 5% HS, accordingly.

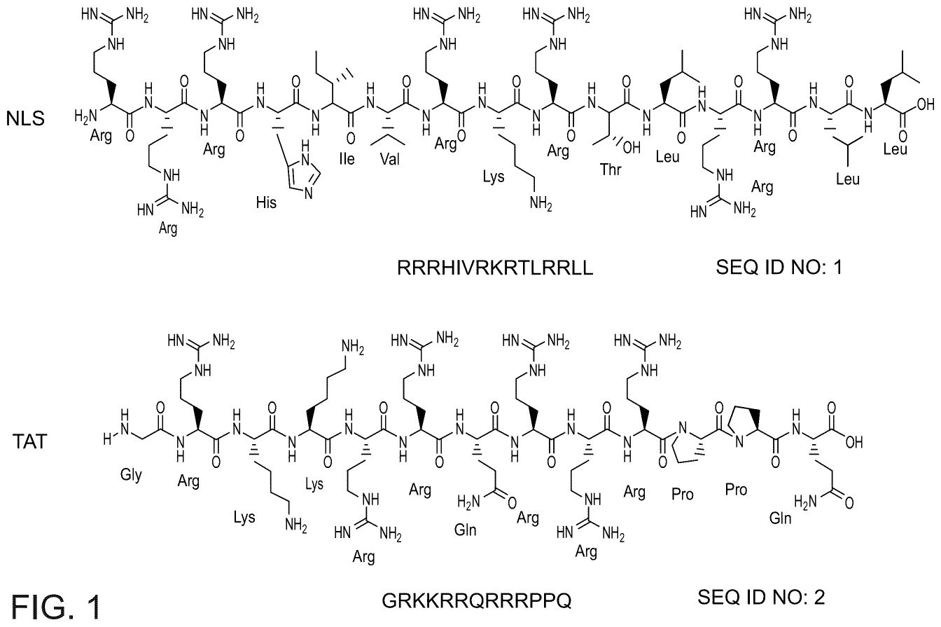

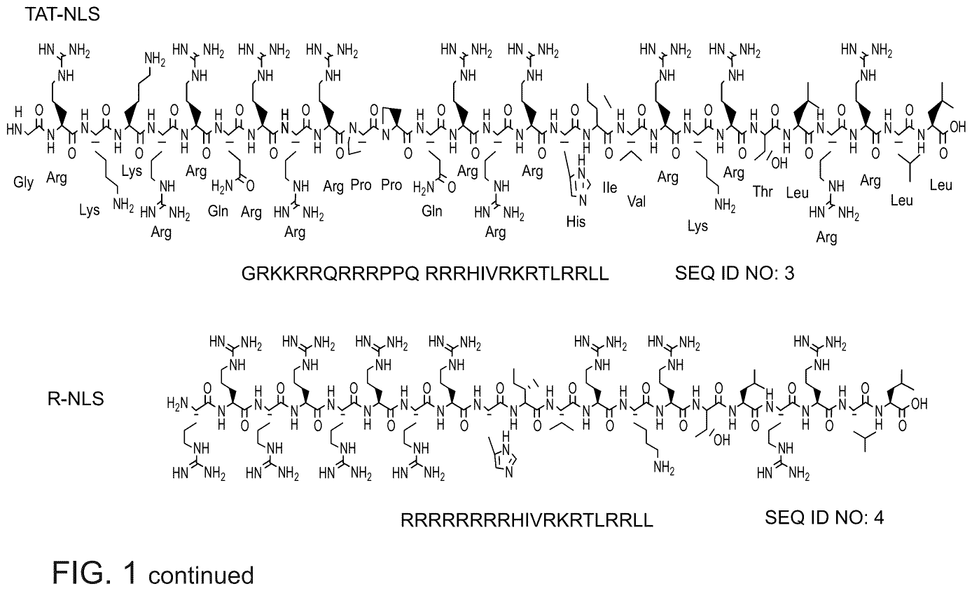

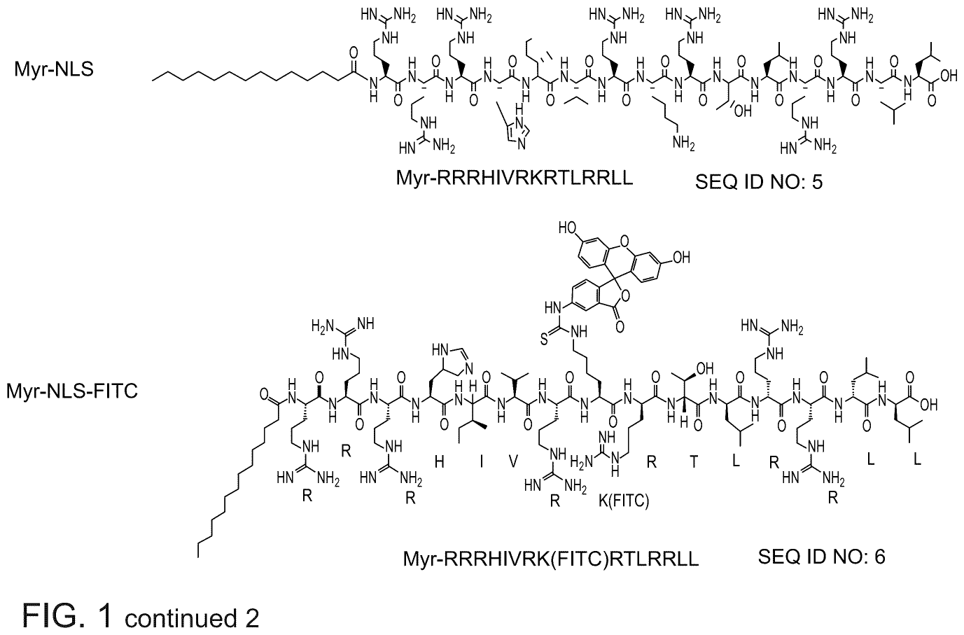

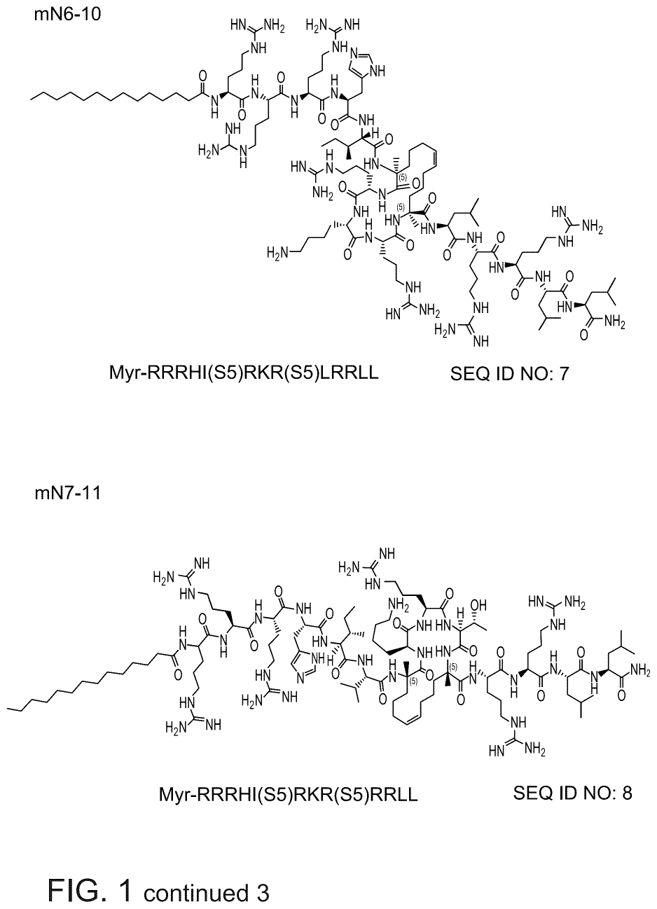

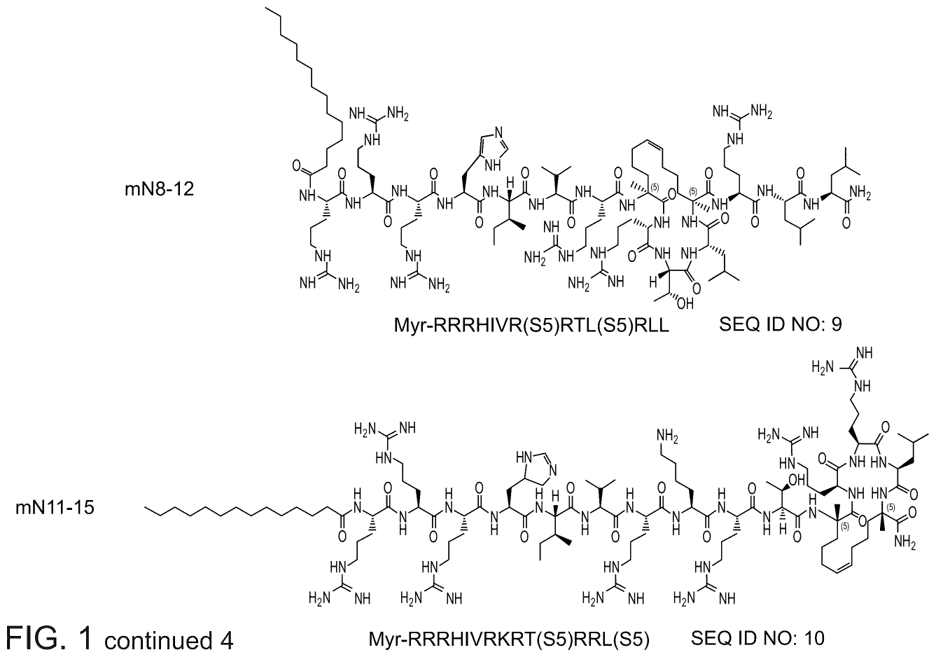

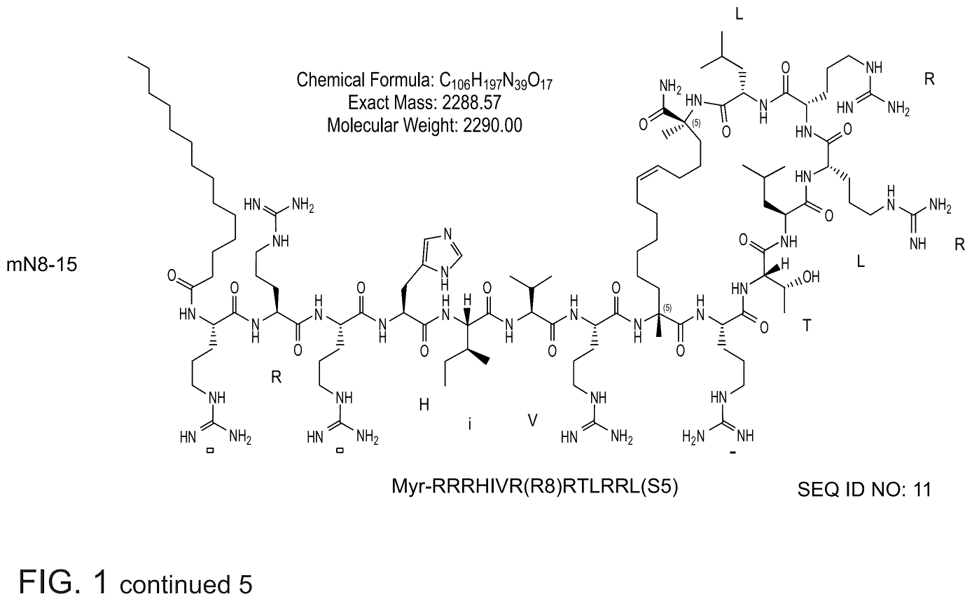

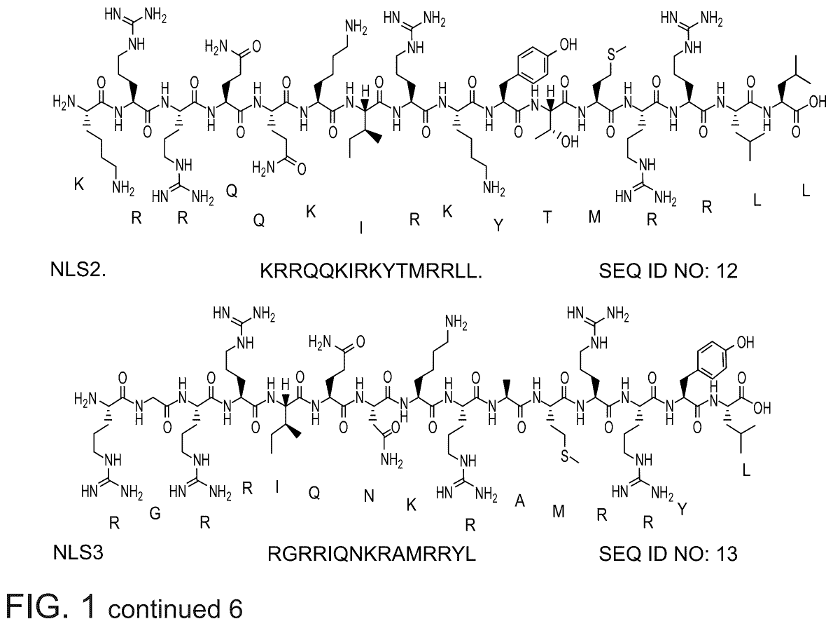

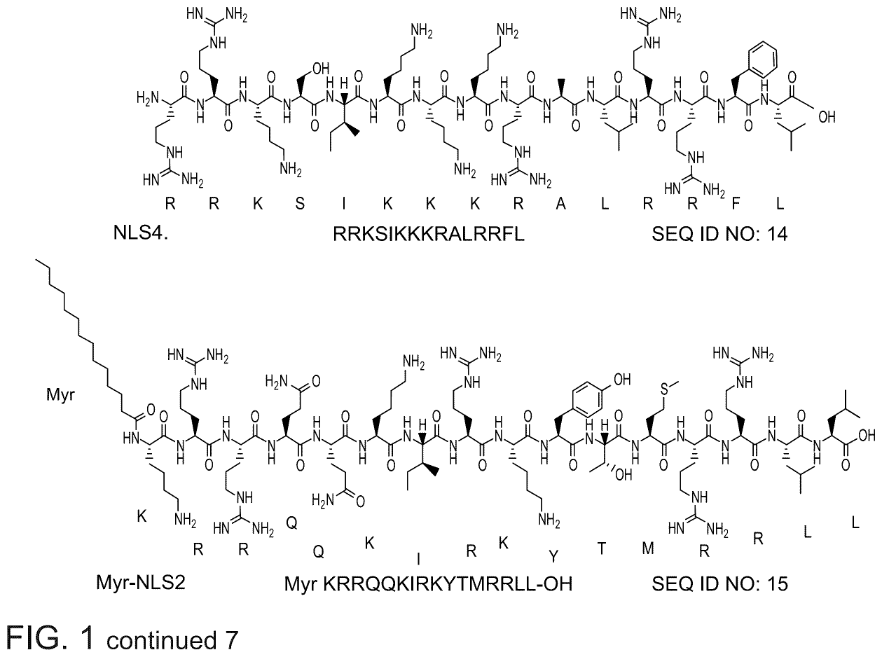

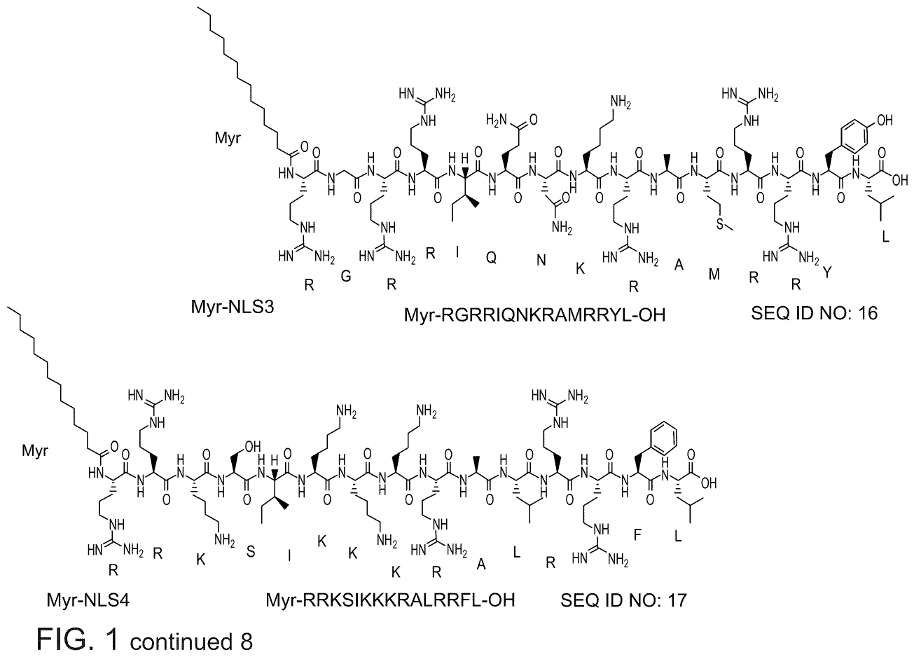

Peptides preparation—All peptides listed in Table 3 and hereinbelow schematically illustrated in FIG. 1 were synthesized by the Blavatnik Center for Drug Discovery (BCDD) in Tel-Aviv University. NLS (SEQ ID NO: 1), TAT (SEQ ID NO: 2), TAT-NLS (SEQ ID NO: 3), R-NLS (SEQ ID NO: 4), myristoylated NLS (referred to herein as myr-NLS or myr-NLS1, SEQ ID NO: 5), myristoylated NLS2 (referred to herein as myr-NLS2 or myr-Pep2, SEQ ID NO: 15), myristoylated NLS3 (referred to herein as myr-NLS3 or myr-Pep3, SEQ ID NO: 16) and myristoylated NLS4 (referred to herein as myr-NLS4 or myr-Pep4, SEQ ID NO: 17) were dissolved in PBS to generate a stock solution of 1.2-1.5 mM. Generation of stapled NLS peptides was effected by either changing amino acids at positions i and i+4 with S-pentenylalanine (S5); or changing amino acids at positions i and i+7 with R-octenylalanine (R8) and S-pentenylalanine respectively. FITC-tagged myristoylated NLS (referred to herein as myr-NLS-FITC, SEQ ID NO: 6) and all stapled myristoylated NLS variants (referred to herein as mN6-10, mN7-11, mN8-12, mN11-15 and mN8-15, SEQ ID Nos: 7-11) were dissolved in DMSO to generate a stock solution of 30 mM. In all experiments PBS and/or DMSO were added to all treatment samples to match PBS/DMSO volume in treatment sample harboring the highest peptide concentration, as a control.

TABLE 3
List of peptides used

| SEQ ID | Peptide name as | | Molecular |
|---|---|---|---|
| NO: 1 | referred to herein | Sequence | formula |
| 1 | NLS | RRRHIVRKRTLRRLL | C₈₇H₁₆₅N₃₉O₁₇ |
| 2 | TAT | GRKKRRQRRRPPQ | C₇₀H₁₃₁N₃₅O₁₆ |
| 3 | TAT-NLS | GRKKRRQRRRPPQRRRHIVRK | C₁₅₇H₂₉₄N₇₄O₃₂ |
| | | RTLRRLL | |
| 4 | R-NLS | RRRRRRRRHIVRKRTLRRLL | |
| 5 | myr-NLS | Myr-RRRHIVRKRTLRRLL-OH | C₁₀₁H₁₉₁N₃₉O₁₈ |
| 6 | myr-NLS-FITC | Myr-RRRHIVRK(FITC)RTLRRLL | C₁₂₂H₂₀₂N₄₀O₃₂S |
| 7 | mN6-10 (i, i + 4 | Myr-RRRHI(S5)RKR(S5)LRRLL | |
| | stapling, | | |
| | changing hydrophobic | | |
| | V6 and hydrophilic T10 | | |
| | to hydrophobic S5) | | |
| 8 | mN7-11 (i, i + 4 | Myr-RRRHIV(S5)KRT(S5)RRLL | |
| | stapling, | | |
| | changing hydrophilic | | |
| | R7 and hydrophobic | | |
| | L11 to hydrophobic S5) | | |
| 9 | mN8-12 (i, i + 4 | Myr-RRRHIVR(S5)RTL(S5)RLL | |
| | stapling, | | |
| | changing hydrophilic | | |
| | K8 and hydrophilic R12 | | |
| | to hydrophobic S5) | | |
| 10 | mN11-15 (i, i + 4 | Myr-RRRHIVRKRT(S5)RRL(S5) | |
| | stapling, changing | | |
| | hydrophobic L11 and | | |
| | L15 to hydrophobic S5) | | |
| 11 | mN8-15 (i, i + 7 | Myr-RRRHIVR(R8)RTLRRL(S5) | |
| | stapling, | | |
| | changing hydrophilic | | |
| | K8 and hydrophobic | | |
| | L15 to hydrophobic R8 | | |
| | and S5 respectively.) | | |
| 12 | NLS2 | KRRQQKIRKYTMRRLL | |
| 13 | NLS3 | RGRRIQNKRAMRRYL | |
| 14 | NLS4 | RRKSIKKKRALRRFL | |
| 15 | myr-NLS2 | Myr-KRRQQKIRKYTMRRLL-OH | C₁₀₈H₁₉₈N₃₆O₂₂S |
| 16 | myr-NLS3 | Myr-RGRRIQNKRAMRRYL-OH | C₉₆H₁₇₄N₃₆O₂₀S |
| 17 | myr-NLS4 | Myr-RRKSIKKKRALRRFL-OH | C₁₀₁H₁₈₈N₃₄0₁₈ |

Methylene blue viability assay—Cells were plated in 96-wells plates at cell densities of 1.5×10³ (EJ, DLD-1), 3×10³ (Rat-1, HCT-116, U87), 5×10³ (SKBR3, MCF7) and 6×10³ (MCF10A) cells/well. On the following day, cells were treated as indicated, and grown for 72 hours. Cell number determination was performed as described previously [8, 16]. IC-50 values were calculated using a non-linear regression model (logarithmic inhibitor vs. normalized response-variable slope) with the GraphPad Prism 6 software.

Lysate preparation and immunoblotting—Cells were seeded at cell densities of 1.5×10⁵ (DLD-1), 1.8×10⁵ (HCT-116) and 2.5×10⁵ (SKBR3) cells/well, treated as indicated and grown for 24 hours. Following, cells were lysed in solubilization buffer; the lysates were cleared by centrifugation and a boiling gel sample buffer was added. Proteins were resolved by SDS-polyacrylamide gel electrophoresis through 10% polyacrylamide gels, and were electrophoretically transferred to nitrocellulose membranes. Membranes were blocked in TBST buffer containing 6% milk, and blotted with primary antibodies. Following, a secondary antibody linked to horseradish peroxidase was added. Immunoreactive bands were detected with the enhanced chemiluminescence reagent Immobilon Crescendo Western HRP substrate (WBLUR0100; Merck Millipore).

Proximity Ligation Assay (PLA)—For proximity ligation assay (PLA) cells were plated in a 16-wells Nunc Lab-Tek glass Chamber Slide System (178599; Thermo Scientific) at a density of 3.5×10³ cells/well. 2 hours post-seeding the cells were treated with 53 μM of either TAT peptide (SEQ ID NO: 2) or TAT-NLS peptide (SEQ ID NO: 3) and grown for 48 hours. Following fixation, cells were incubated with rabbit anti-ErbB2 and mouse anti-nucleolin antibodies. PLA was performed using the Duolink In-Situ PLA probes: anti-rabbit MINUS and anti-mouse PLUS, and the Duolink In-Situ Detection Reagents Red kit (DUO92005; DUO92001; DUO92008, respectively; Sigma-Aldrich), according to the manufacturer's instructions. Nuclei were stained using the Duolink In-Situ Mounting Medium with DAPI (DUO82040; Sigma-Aldrich). Slides were visualized 24 hours post-staining and images were obtained using an Olympus motorized inverted research microscope Model IX81 (60× magnification). Signal intensity was determined using ImageJ software.

Peptide penetration assay—MCF7 cells were seeded onto poly-L-lysine coated 22 mm glass cover slips at a density of 10⁵ cells/cover slip. The following day cells were treated with the myr-NLS-FITC peptide (SEQ ID NO: 6) as indicated. Following, cells were incubated for 10 minutes with the membrane-permeable fluorescent DNA dye bisbenzimide (Hoechst 33342, 1 μg/ml; Sigma-Aldrich), washed twice in PBS and fixed for 30 minutes in 4% paraformaldehyde (PFA). Cells were then washed three times in PBS. Slides were imaged using the Leica TCS SP8 confocal microscope (×63 magnification).

Colony formation assay—Cells were plated onto 12-well plate (6×10⁴ cells/well) and treated with either PBS or myr-NLS at the IC50 concentration for 3 days. Cells were collected and replated in 10-cm plates (at 3 dilutions of 1:10, 1:20 and 1:40). After 7-14 days when visible and yet separated colonies were formed, the cells were fixed with 0.1% acetic acid in PBS, and stained with 0.4% crystal violet in 0.1% acetic acid. Total colonies area was calculated using the ImageJ program.

Pancreatic Tumor Growth in Nude Mice—The study was conducted according to the NIH Guidelines for Use and Care of Laboratory Animals and following the approval by Animal Care Committee of the Tel Aviv University. Nude CD1-Nu mice (25-30 g) housed in barrier facilities on a 12-h light/dark cycle one week prior to the injection of tumor cells (day zero). On day zero, 2×10⁶ Panc 1 cells in 0.1 ml of PBS were implanted subcutaneously just above the right femoral joint. Tumor size was measured every 3-4 days. When the tumors were apparently seen and the calculated tumor size was 40 mm³, the animals were divided randomly into two groups of mice (control vehicle treated and myr-NLS (SEQ ID NO: 5) treated; 8 mice per group). Treatment with the myr-NLS (SEQ ID NO: 5) was performed by intraperitoneal injection of 25 μg in 100 μl PBS (or 100 μl PBS for control mice) every day. Tumors size was measured 3 times a week. At the end of the experiment, the mice were sacrificed, and the tumors were dissected and used for further analysis.

Colon Tumor Growth in Nude Mice—Nude CD1-Nu mice (25-30 g) housed in barrier facilities on a 12-h light/dark cycle one week prior to the injection of tumor cells (day zero). On day zero, 5×10⁶ DLD-1 cells were implanted subcutaneously just above the right femoral joint. Tumor size was measured every 3-4 days. When the tumors were apparently seen, the animals were divided randomly into three groups of mice [control vehicle treated, myr-NLS1 (SEQ ID NO: 5), myr-NLS4 (SEQ ID NO: 17)]. Treatment was performed by intraperitoneal injection of 25 μg in 100 μl PBS every day. Mice were weighed and tumors size was measured 3 times a week. At the end of the experiment, the mice were sacrificed, and the tumors were dissected and used for further analysis.

Western blot analysis—Half of each tumor was homogenized in lysis buffer, using a Potter-Elvehjem manual homogenizer, cleared and kept at −70° C. The lysates (30 μg) were resolved by SDS-polyacrylamide gel electrophoresis (SDS-PAGE) and transferred to nitrocellulose membranes. The membranes were blocked with TBST buffer containing 6% milk and reacted with the indicated primary antibodies followed by second antibodies. Immunoreactive bands were detected with the enhanced chemiluminescence reagent (Luminata Crescendo Western HRP). Densitometric quantification analysis of the resulted images was done using ImageJ program.

Statistical analysis—All experiments were performed at least three times. Results are presented as means±SD/SE. Differences between means were assessed by the 1-tailed Student's t-test, one-way ANOVA or regression analysis. Significance was assigned at p<0.05.

### Example 1

### Peptides Comprising the NLS Sequence of ERBB1 Decrease Viability of Cancerous Cells In-Vitro

The present inventors have generated peptides comprising the nuclear localization sequence (NLS) of ErbB1, which is the ErbB1 interacting domain with nucleolin (see Table 3 hereinabove and FIG. 1). In addition, some of the peptides were fused to a TAT sequence, a poly R sequence or modified with myristoyl, in order to increase peptide penetration into cells.

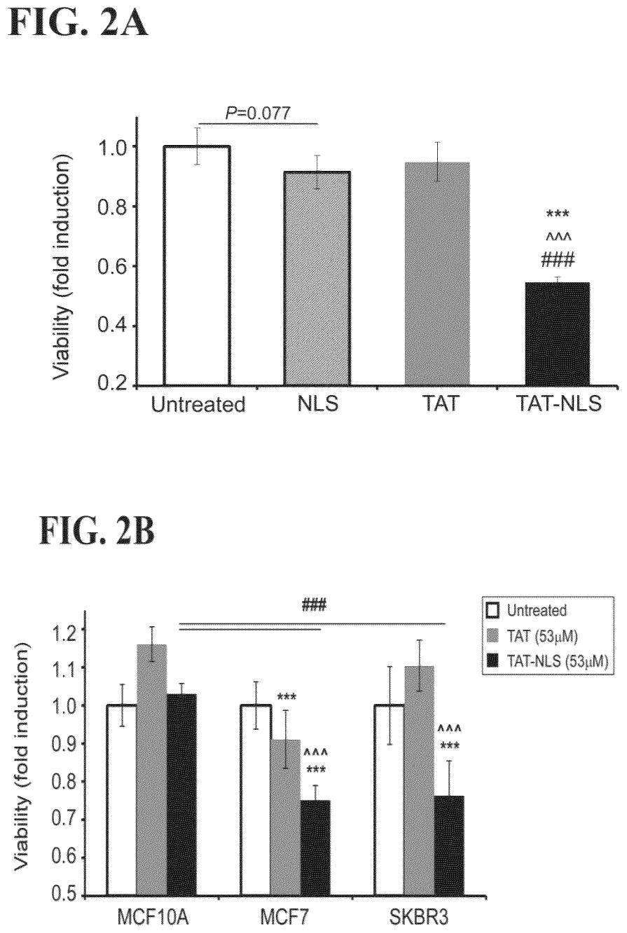

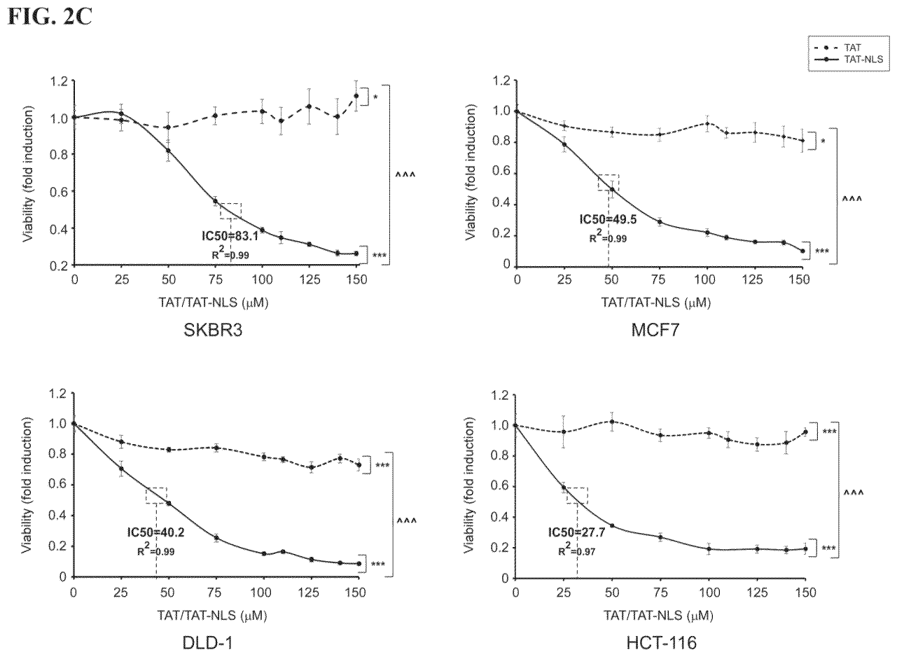

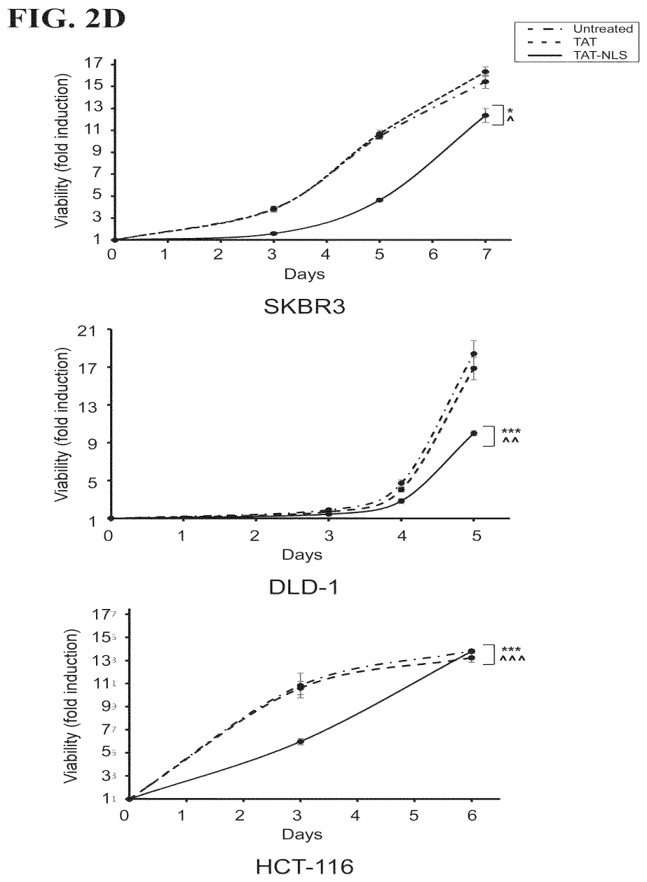

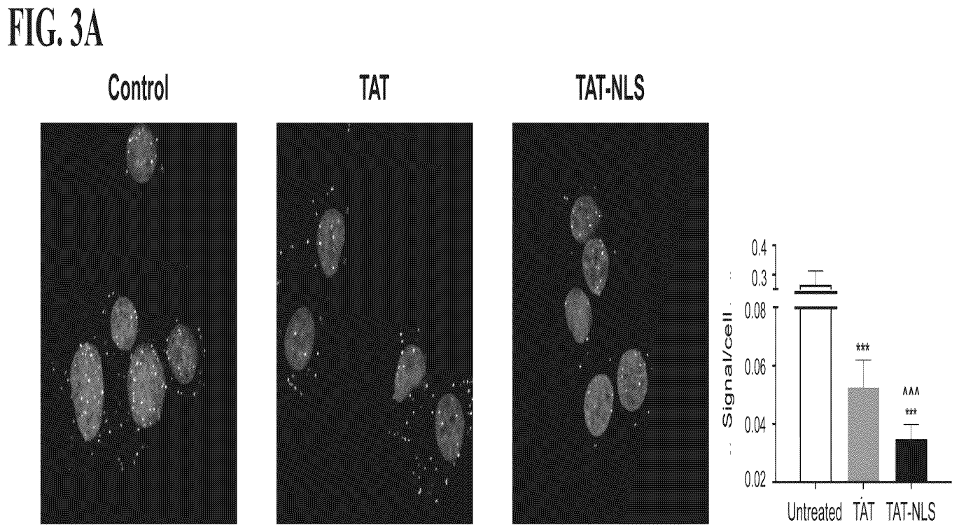

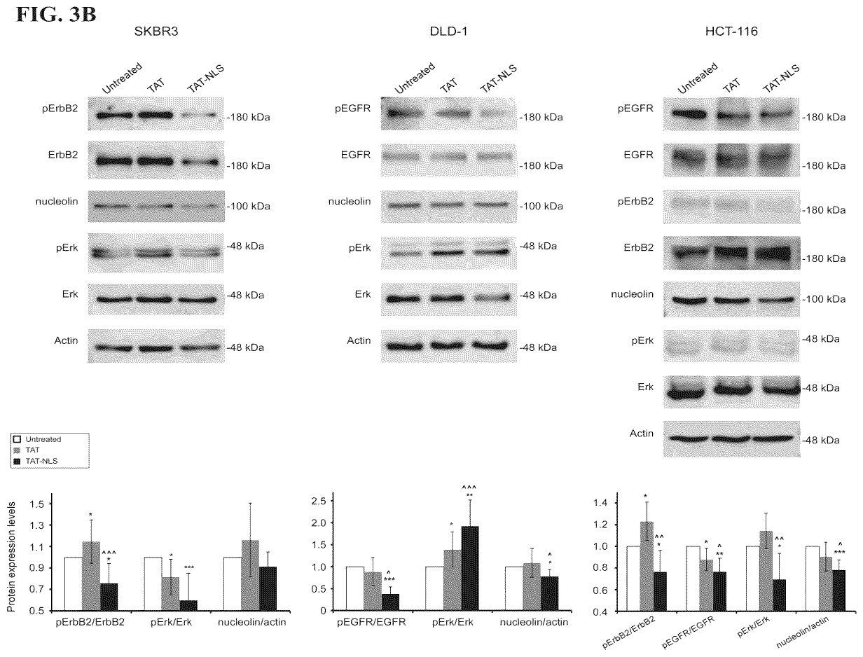

In the first stage, the NLS peptide (SEQ ID NO: 1) was fused to a TAT sequence (SEQ ID NO: 2) to generate a TAT-NLS peptide (SEQ ID NO: 3). As shown in FIGS. 2A-C, the TAT-NLS peptide (SEQ ID NO: 3), but not the TAT or NLS peptides (SEQ ID NO: 2 or 1, respectively), inhibited cell growth of DLD-1 and HCT-116 colon cancer cells; and SKBR3 and MCF7 breast cancer cells, while not affecting viability of the non-cancerous cells MCF10. As shown in FIG. 2C, the IC50 values of the TAT-NLS peptide (SEQ ID NO: 3) on cell viability of the four cancer cell lines SKBR, MCF7, DLD-1 and HCT116 were 83.1, 49.5, 40.2 and 27.7 PM, respectively. In addition, treatment with the TAT-NLS peptide (SEQ ID NO: 3) reduced the interaction between the ErbB2 receptor and nucleolin as determined by a proximity ligation assay (PLA, see FIG. 3A) and inhibited ErbB1 and ErbB2 phosphorylation in SKBR3, DLD-1 and HCT116 cancer cell lines (FIG. 3B), indicating that the peptide reduced activation of ErbB receptors.

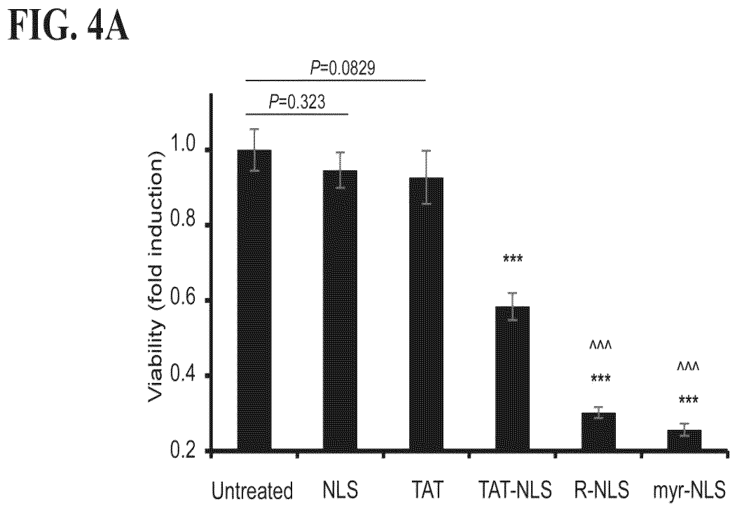

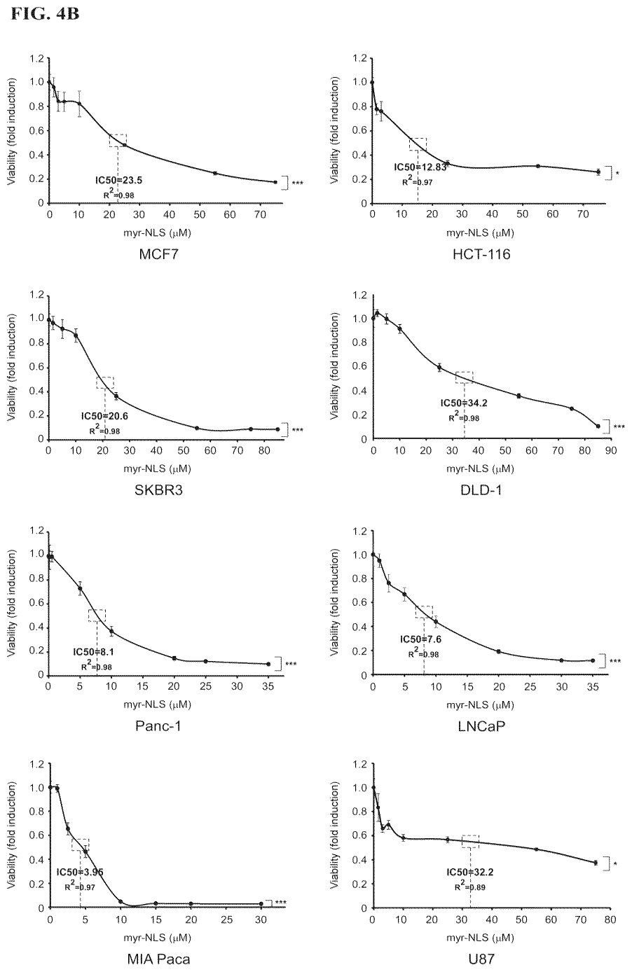

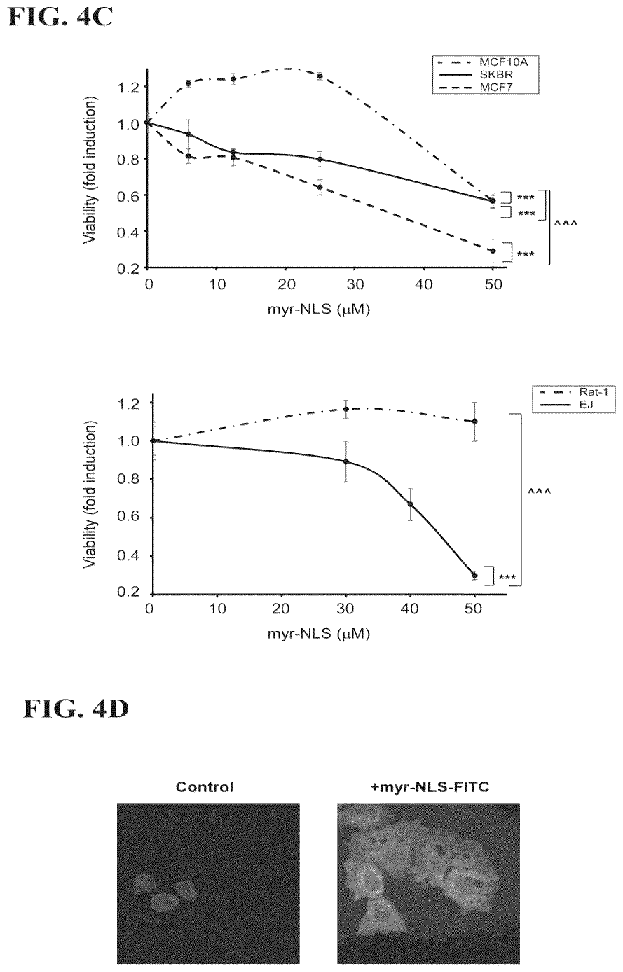

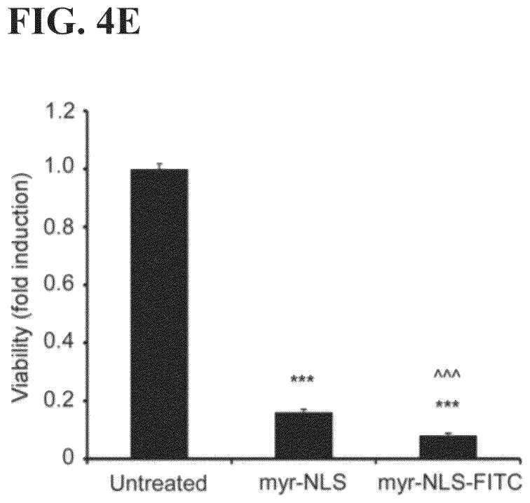

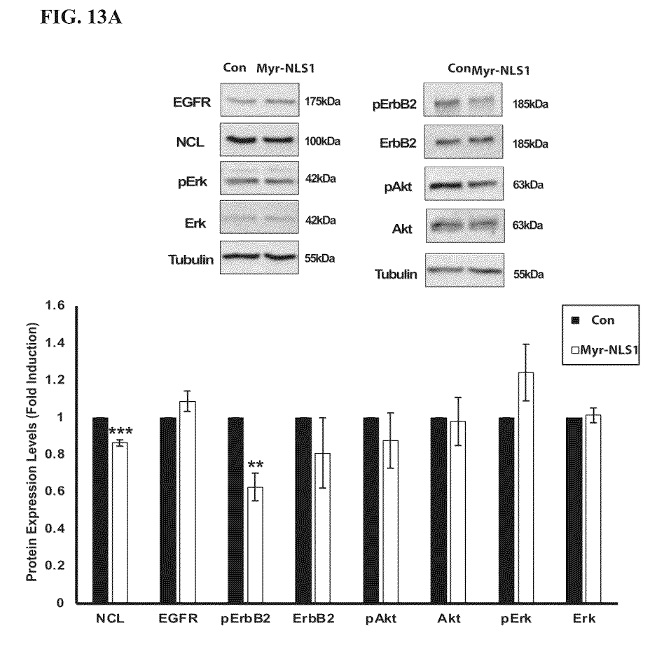

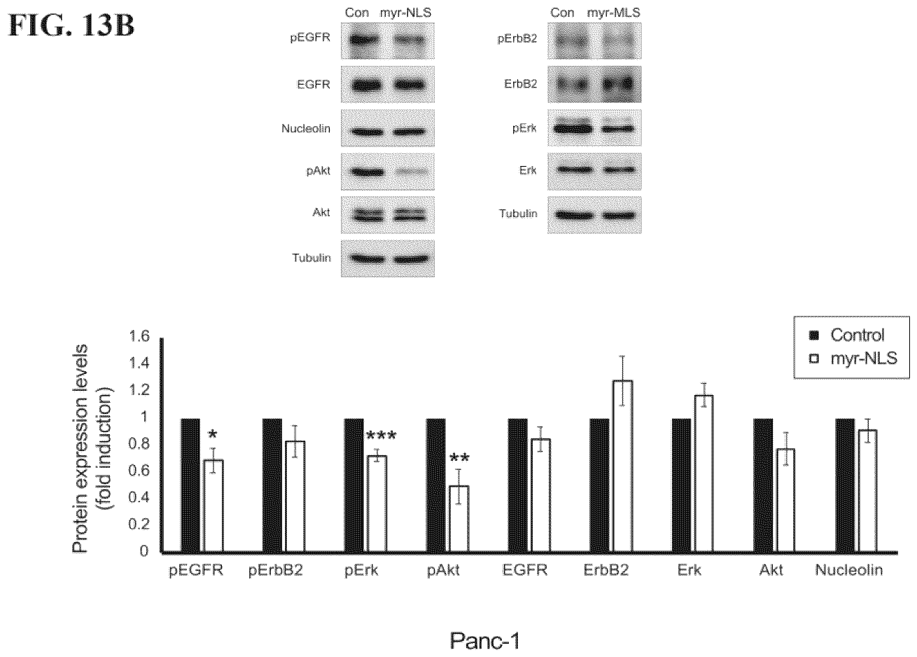

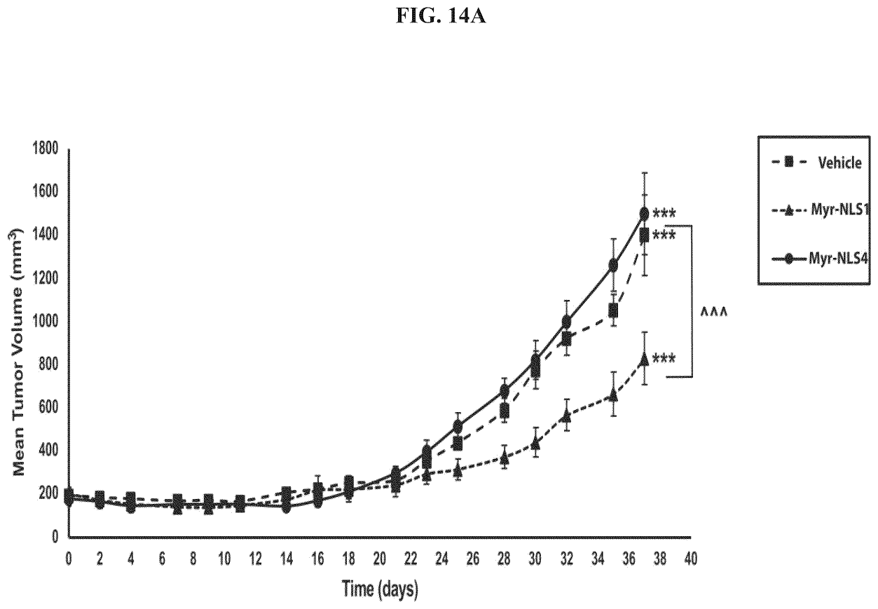

In the next step, two other modifications were introduced to the NLS peptide (SEQ ID NO: 1): addition of Poly R sequence (R-NLS peptide, SEQ ID NO: 4) as well as myristoylation (addition of myristoyl (myr-NLS, SEQ ID NO: 5). Both these modified peptides significantly reduced viability of SKBR3 breast cancer cells (FIG. 4A). As shown in FIGS. 4B and 4C, The IC50 values of the myr-NLS peptide (SEQ ID NO: 5) in MCF7, SKBR3, HCT116, DLD-1, LNCaP, Panc 1, MiaPaCa2 and U87 cell lines were 23.5, 20.6, 12.83, 34.2, 7.6, 8.1, 3.95 and 32.2, respectively, indicating that the myr-NLS was even more efficient than the TAT-NLS peptide. In addition, the effect of the myr-NLS peptide (SEQ ID NO: 5) was much more pronounced on viability of the breast cancer cell lines, MCF7 and SKBR3, as compared to non-transformed MCF10 cells (FIG. 4C). Moreover, the myr-NLS peptide (SEQ ID NO: 5) decreased viability of Rat-1 fibroblast cells transformed with constitutive Ras (EJ) while it had no effect on the non-transformed Rat-1 cells (FIG. 4C). In order to visualize entry of the peptide into the cells, a FITC-conjugated myr-NLS (myr-NLS-FITC; FIG. 1, SEQ ID NO: 6) was synthesized. As shown in FIG. 4D, 1 hour following incubation with 7.5 μM myr-NLS-FITC, the peptide was observed in the cells. Of note, FITC-conjugation did not affect myr-NLS activity (FIG. 4E). Moreover, treatment of DLD-1 or Panc-1 cells for 24 hours with the myr-NLS peptide (SEQ ID NO: 5), reduced the levels of expression of nucleolin and phosphorylated EGFR (FIGS. 13A-B).

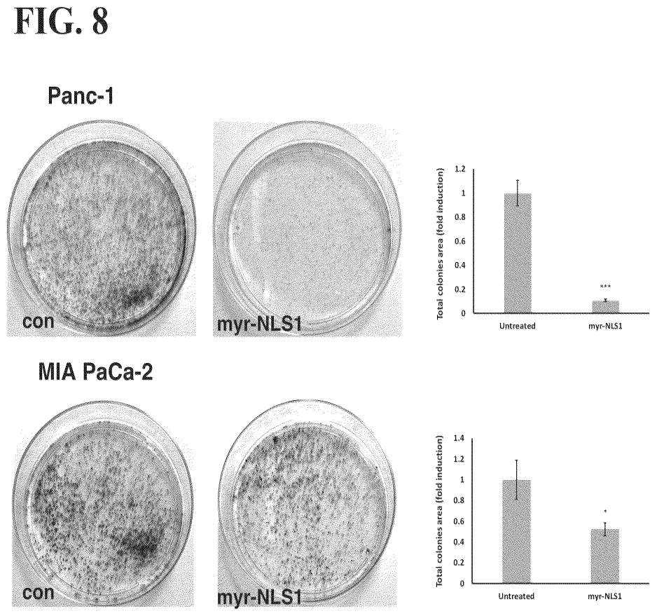

In order to test the effect of myr-NLS on cell tumorigenicity, a colony formation assay was performed. To this end, Panc-1 and MIA PaCa-2 cells were treated or untreated for 72 hours with IC50 dose of myr-NLS (SEQ ID NO: 5, 8 μM and 4.5 μM respectively). Cells were then seeded at the same dilutions in new plates until colonies were formed. As shown, treated cells formed fewer and smaller colonies than untreated cells, as evident by total colonies area quantification (FIG. 8).

### Example 2

### Stapled Peptides Comprising the NLS Sequence of ERBB1 Decrease Cells' Viability In-Vitro

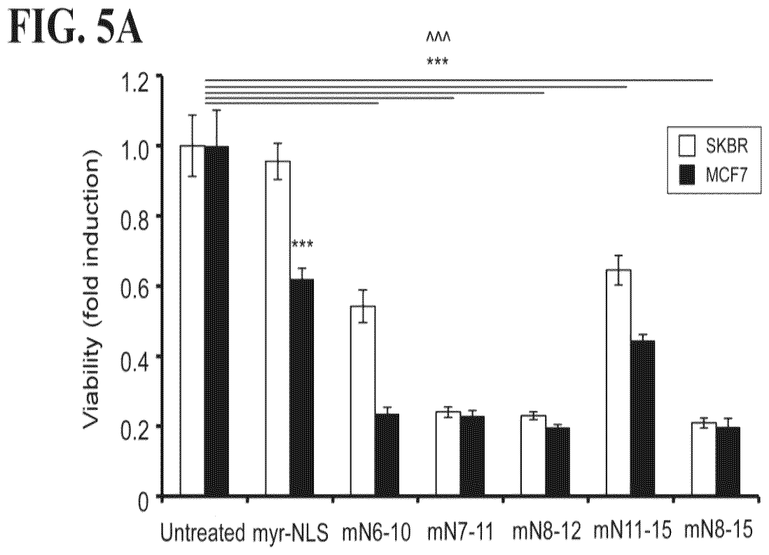

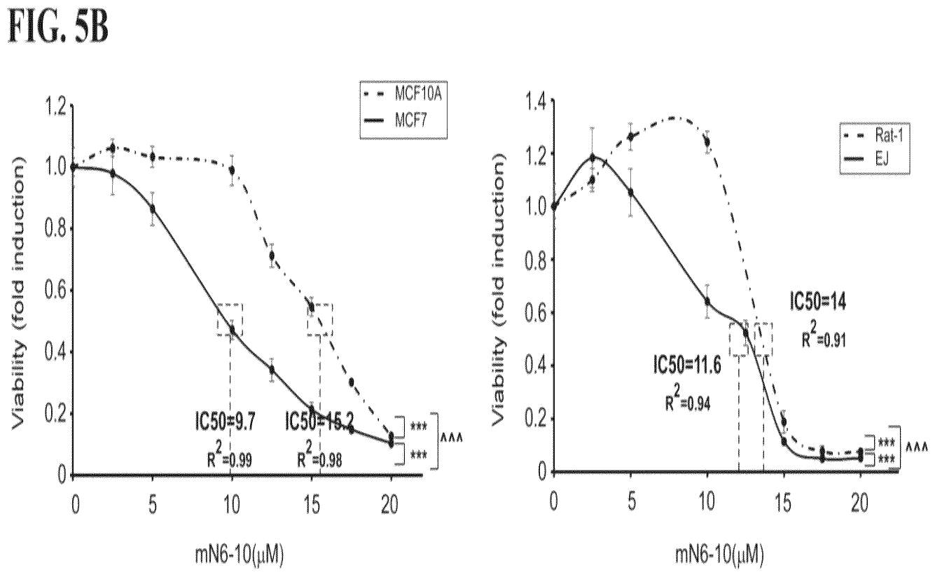

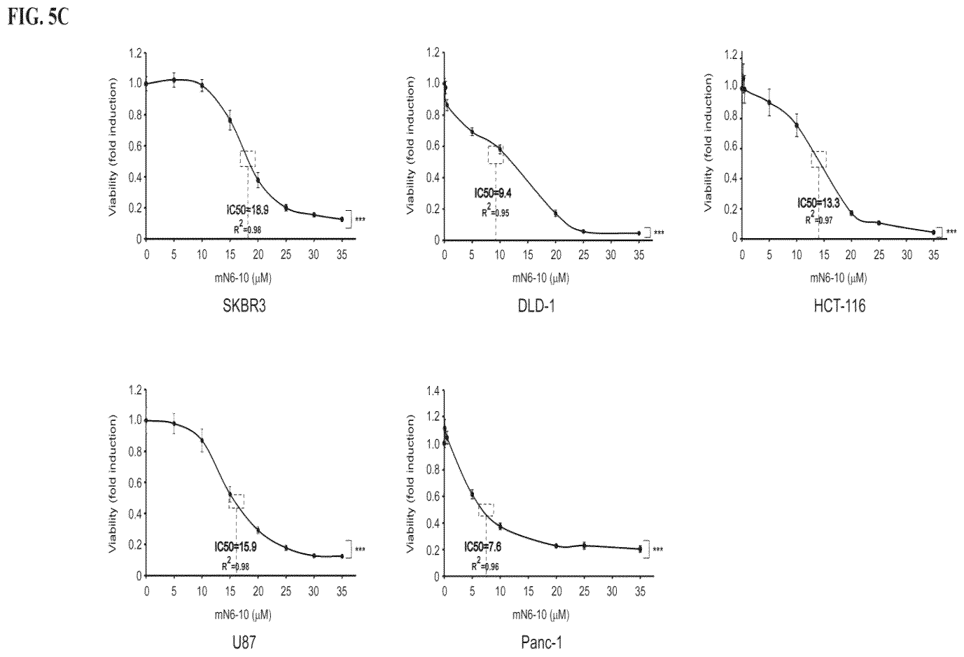

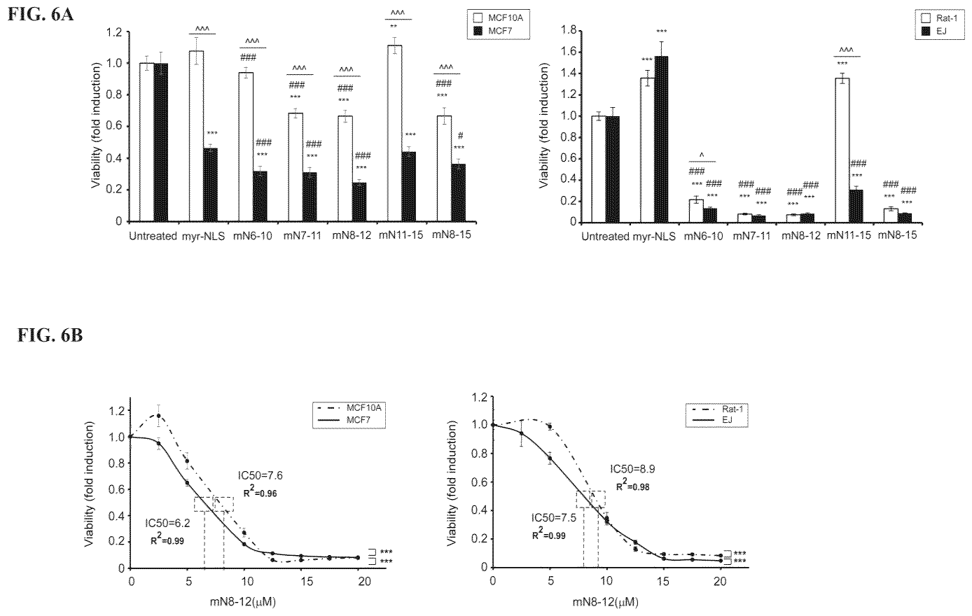

The present inventors have generated various stapled peptides based on the myr-NLS peptide (FIG. 1) and evaluated their effect on viability of cancerous and non-cancerous cells lines. As shown in FIG. 5A, all the stapled peptides reduced viability of SKBR3 and MCF7 breast cancer cells. As an example, the mN6-10 peptide (SEQ ID NO: 7) was even more efficient than the myr-NLS peptide (SEQ ID NO: 5) in the tested SKBR3 and MCF7 breast cancer cells; thus, its effect on several other cancer cell lines was tested (FIGS. 5B-C). As shown, the IC50 values were 18.9, 9.4, 13.3, 15.9, 9.7, 7.6 and 11.6 mM in SKBR3, DLD-1, HCT116, U87, MCF7, Panc-1 and EJ cells, respectively. Next, the effect of the stapled peptides on viability of the breast cancer MCF7 cells compared to the non-cancerous MCF10 breast cells was evaluated. As shown in FIGS. 6A-B, the stapled peptides mN6-10 (SEQ ID NO: 7) and mN11-15 (SEQ ID NO: 10) decreased MCF7 cell viability and had no effect on the MCF10 cells. Other stapled peptides such as mN8-12 (SEQ ID NO: 9) decreased viability of both cell lines (FIG. 6B). Taken together, a stapled modification may improve the efficiency of the peptide.

### Example 3

a Combined Treatment with a Peptide Comprising the NLS Sequence of ERBB1 and an Anti-Nucleolin Agent

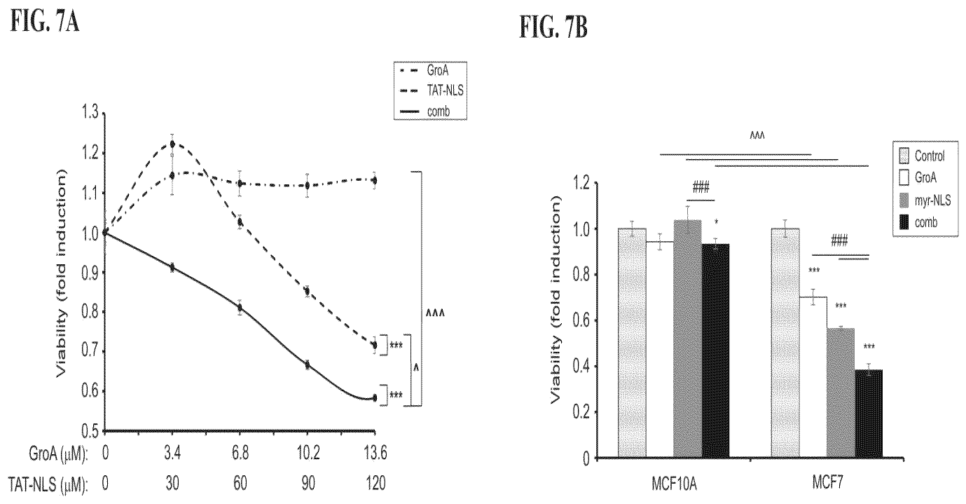

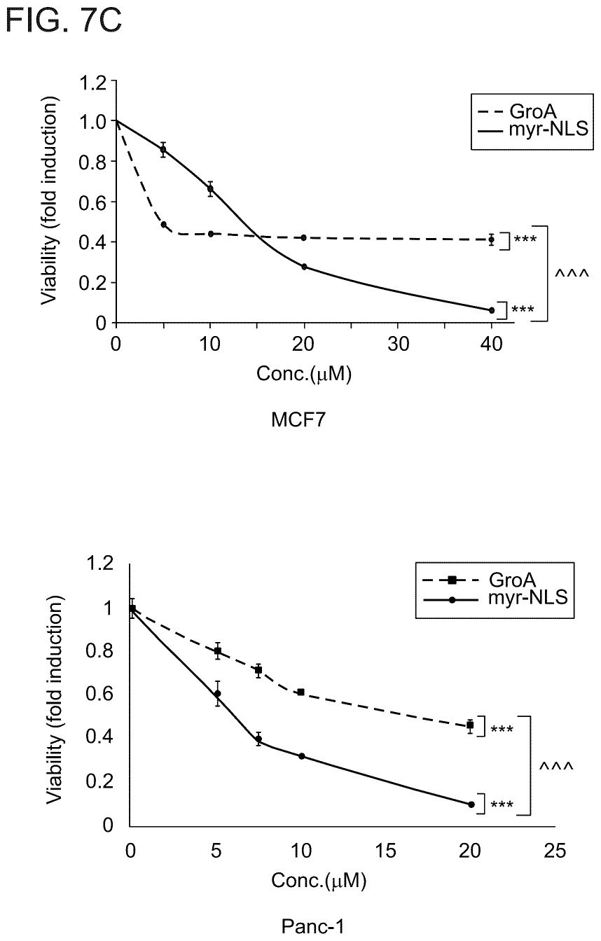

The effect of the generated peptides in a combined treatment with a known anti-nucleolin G-rich oligonucleotide, GroA (AS1411) was evaluated. As shown in FIGS. 7A-C, the combined treatment was significantly more effective compared to treatment with each of them alone.

### Example 4

### Peptides Comprising the NLS Sequence of ERBB1 Decrease Viability of Cancerous Cells In-Vivo

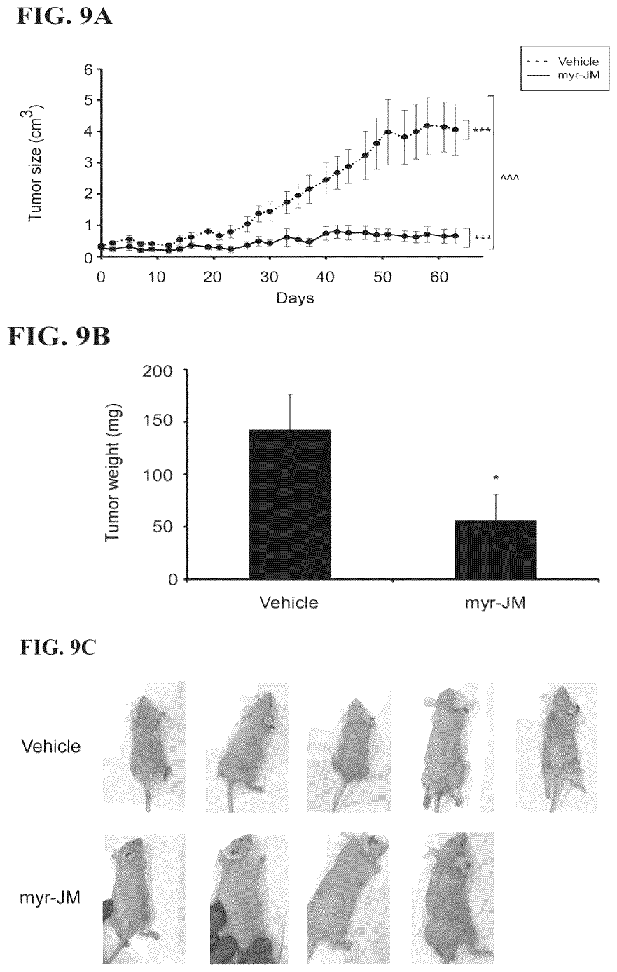

The in-vivo anti-tumor effect of the generated peptides was evaluated in nude mice implanted with the pancreatic tumor cell line Panc-1 or with the colon cancer line DLD-1. As shown in FIGS. 9A-C and 14A-C, treatment with the myr-NLS peptide (SEQ ID NO: 5) significantly reduced tumor cell growth in both tumor models.

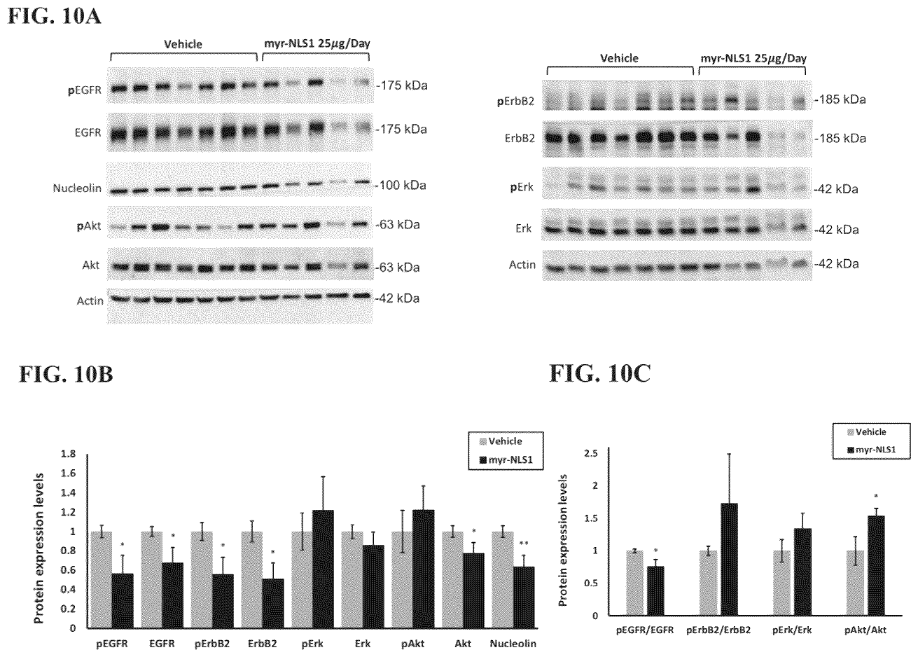

Following, the present inventors examined whether the reduction of the tumor growth rate correlates with alterations expression and activation of the relevant proteins. To this end, lysates were prepared form the dissected tumors for protein levels evaluations. The tumor lysates were processed for western blot analysis (30 μg protein, 10% SDS polyacrylamide gel electrophoresis) and protein levels were determined using the indicated antibodies. Treatment reduced the expression levels of EGFR and the levels of phosphorylated EGFR (FIGS. 10A-C). It also reduced the levels of total ErbB2 and phosphorylated ErbB2 expression (FIGS. 10A-C). The ratio between EGFR and its phosphorylation state indicated that treatment affected the activation of the EGFR (FIG. 10C). A significant reduction in nucleolin and Akt levels was also indicated following treatment with myr-NLS (FIGS. 10A-C).

### Example 5

Peptides Comprising the NLS Sequence of ErbB/her Family Receptors Decrease Viability of Cancerous Cells In-Vitro and in-Vivo

The results obtained with the generated peptides comprising the NLS of ErbB1 attached to a cell penetrating moiety (e.g. TAT sequence, a poly R sequence or modified with myristoyl) prompted the present inventors to test the anti-cancer effect of peptides comprising the NLS of other members of the ErbB/HER family of receptors. To this end, myristoylated peptides comprising the NLS sequences of ErbB2, ErbB3 or ErbB4 were synthesized, namely myr-NLS2 (SEQ ID NO: 15), myr-NLS3 (SEQ IS NO: 16) and myr-NLS-4 (SEQ ID NO: 17), respectively.

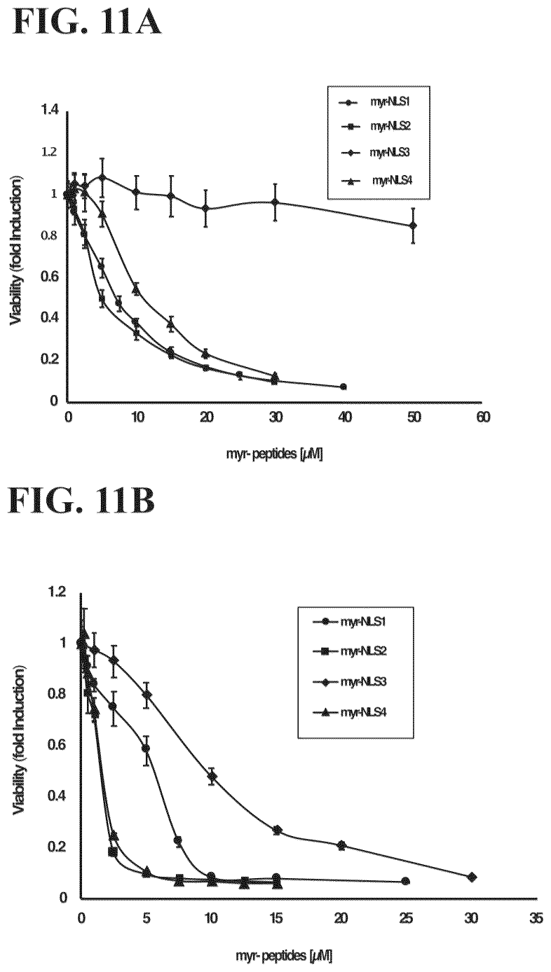

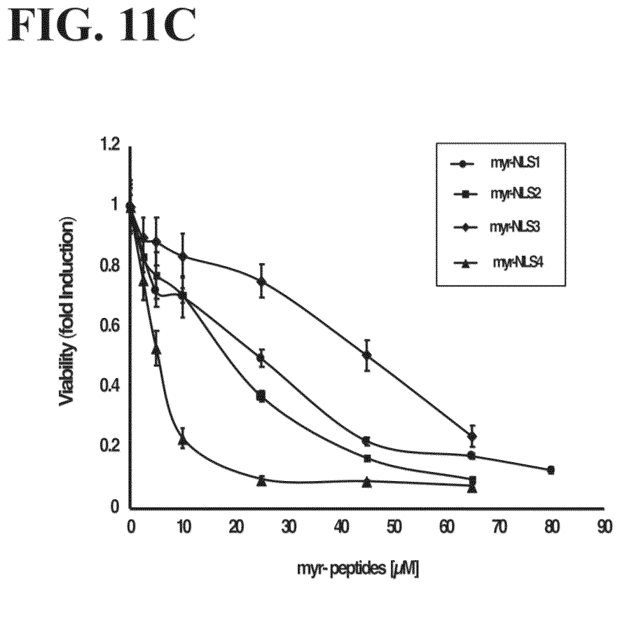

The effect of the peptides on viability of cancer cells was tested in-vitro in dose response assays in three cell lines, Panc-1, MIA PacA2 and DLD-1 (FIGS. 11A-C). As shown, all tested myrisloylated peptides had an anti-tumor effect on the tested cell lines.

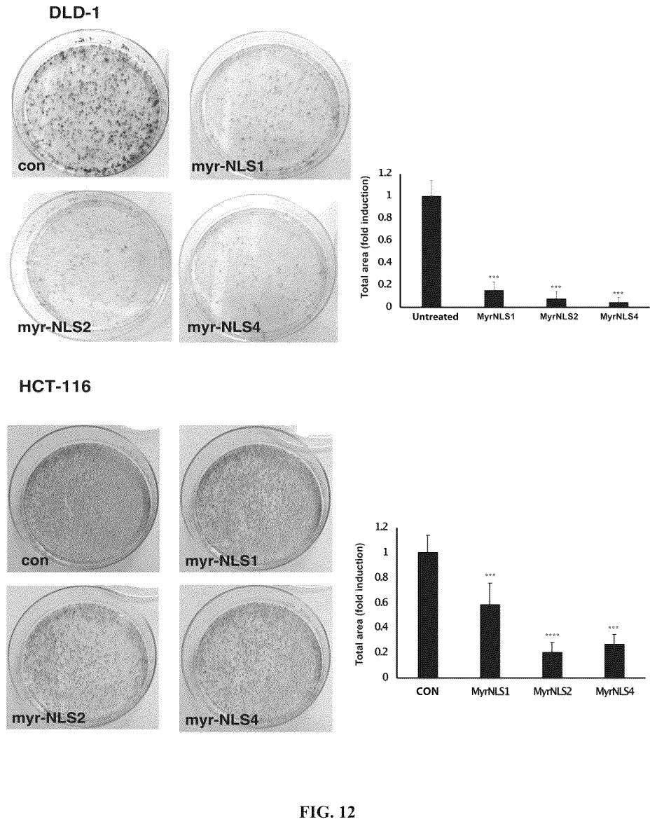

In addition, a colony formation assay was conducted to further examine the anti-tumor effect of the myr-NLS (SEQ ID NO: 5), myr-NLS2 (SEQ ID NO: 15) and myr-NLS4 (SEQ ID NO: 17) peptides. To this end, DLD-1 cells were treated or untreated for 72 hours with 20 μM of the myr-peptides. Cells were then seeded at the same dilutions in new plates until colonies were formed. Cells treated with each of the myr-peptides formed fewer and smaller colonies than untreated cells, as evident by total colonies area quantification (FIG. 12).

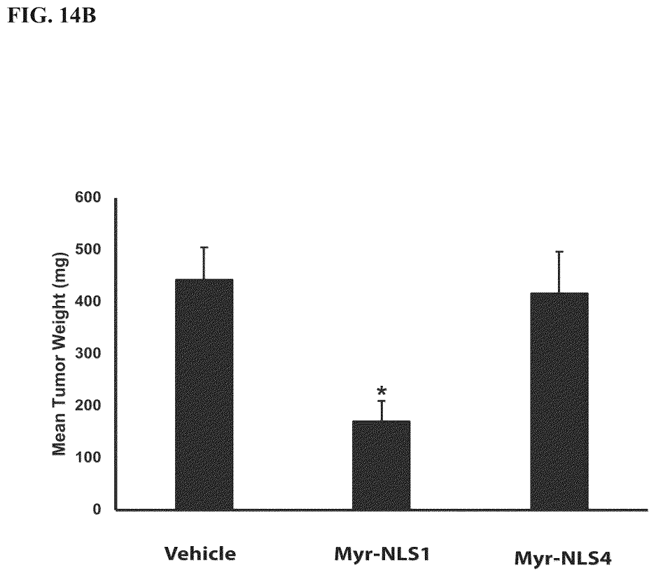

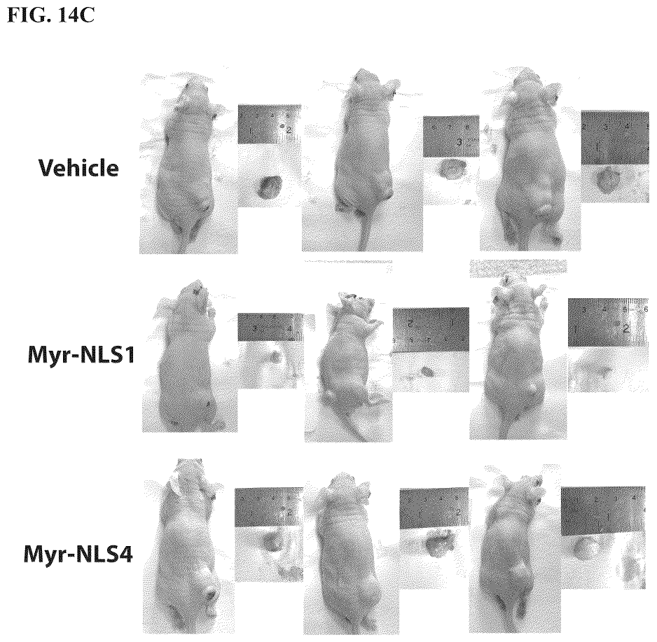

Following, the in-vivo anti-tumor effect of the generated peptides was evaluated in nude mice implanted with the colon cancer line DLD-1. As shown in FIGS. 14A-C, treatment with the myr-NLS peptide (SEQ ID NO: 5) or the myr-NLS4 (SEQ ID NO: 17) significantly reduced tumor cell growth in the tested tumor model.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Holbro T, Hynes N. ErbB receptors: directing key signaling networks throughout life. Annu Rev Pharmacol Toxicol. 2004; 44:195-217.
2. Spencer K S, Graus-Porta D, Leng J, Hynes N E, Klemke R L. ErbB2 is necessary for induction of carcinoma cell invasion by ErbB family receptor tyrosine kinases. J Cell Biol. 2000; 148(2):385-97. PubMed PMID: 10648571; PubMed Central PMCID: PMC2174273.
3. Ferlay J, Soerjomataram I, Dikshit R, Eser S, Mathers C, Rebelo M, et al. Cancer incidence and mortality worldwide: sources, methods and major patterns in GLOBOCAN 2012. International journal of cancer. 2015; 136(5):E359-86. doi: 10.1002/ijc.29210. PubMed PMID: 25220842.
4. Bates P J, Kahlon J B, Thomas S D, Trent J O, Miller D M. Antiproliferative activity of G-rich oligonucleotides correlates with protein binding. J Biol Chem. 1999; 274(37):26369-77. PubMed PMID: 10473594.
5. Srivastava M, Pollard H B. Molecular dissection of nucleolin's role in growth and cell proliferation: new insights. FASEB J. 1999; 13:1911-22.
6. Di Segni A, Farin K, Pinkas-Kramarski R. Identification of nucleolin as new ErbB receptors-interacting protein. PLoS ONE. 2008; 3(6):e2310. PubMed PMID: 18523588.
7. Farin K, Schokoroy S, Haklai R, Cohen-Or I, Elad-Sfadia G, Reyes-Reyes M E, et al. Oncogenic Synergism between ErbB1, Nucleolin, and Mutant Ras. Cancer Res. 2011; 71(6):2140-51. Epub 2011/01/25. doi: 0008-5472.CAN-10-2887 [pii]10.1158/0008-5472.CAN-10-2887. PubMed PMID: 21257709.
8. Wolfson E, Solomon S, Schmukler E, Goldshmit Y, Pinkas-Kramarski R. Nucleolin and ErbB2 inhibition reduces tumorigenicity of ErbB2-positive breast cancer. Cell Death Dis. 2018; 9(2):47. doi: 10.1038/s41419-017-0067-7. PubMed PMID: 29352243.
9. Wolfson E, Goldenberg M, Solomon S, Frishberg A, Pinkas-Kramarski R. Nucleolin-binding by ErbB2 enhances tumorigenicity of ErbB2-positive breast cancer. Oncotarget. 2016; 7(40):65320-34. doi: 10.18632/oncotarget.11323. PubMed PMID: 27542246; PubMed Central PMCID: PMC5323158.
10. Farin K, Di Segni A, Mor A, Pinkas-Kramarski R. Structure-function analysis of nucleolin and ErbB receptors interactions. PLoS One. 2009; 4(7):e6128. PubMed PMID: 19578540.
11. Goldshmit Y, Schokoroy Trangle S, Afergan F, Iram T, Pinkas-Kramarski R. Nucleolin inhibitor GroA triggers reduction in epidermal growth factor receptor activation: Pharmacological implication for glial scarring after spinal cord injury. J Neurochem. 2016; 138(6):845-58. doi: 10.1111/jnc.13730. PubMed PMID: 27399849.
12. Goldshmit Y, Trangle S S, Kloog Y, Pinkas-Kramarski R. Interfering with the interaction between ErbB1, nucleolin and Ras as a potential treatment for glioblastoma. Oncotarget. 2014; 5(18):8602-13. Epub 2014/09/28. PubMed PMID: 25261371; PubMed Central PMCID: PMC4226707.
13. Schokoroy S, Juster D, Kloog Y, Pinkas-Kramarski R. Disrupting the Oncogenic Synergism between Nucleolin and Ras Results in Cell Growth Inhibition and Cell Death. PLoS One. 2013; 8(9):e75269. PubMed PMID: 24086490.
14. Varadan V, Gilmore H, Miskimen K L, Tuck D, Parsai S, Awadallah A, et al. Immune Signatures Following Single Dose Trastuzumab Predict Pathologic Response to Preoperative Trastuzumab and Chemotherapy in HER2-Positive Early Breast Cancer. Clin Cancer Res. 2016; 22(13):3249-59. doi: 10.1158/1078-0432.CCR-15-2021. PubMed PMID: 26842237; PubMed Central PMCID: PMC5439498.
15. Bates P J, Choi E W, Nayak L V. G-rich oligonucleotides for cancer treatment. Methods in molecular biology (Clifton, NJ. 2009; 542:379-92. PubMed PMID: 19565914.
16. Schmukler E, Grinboim E, Schokoroy S, Amir A, Wolfson E, Kloog Y, et al. Ras inhibition enhances autophagy, which partially protects cells from death. Oncotarget. 2013; 4(1):142-52. Epub 2013/07/13. PubMed PMID: 23847721; PubMed Central PMCID: PMC3702214.

1. A composition of matter comprising a peptide comprising an amino acid sequence of a nuclear localization sequence (NLS) of a HER receptor capable of binding a nucleolin polypeptide, said peptide being attached to a cell penetrating moiety.

2. (canceled)

3. The composition of matter of claim 1, wherein said cell penetrating moiety comprises a myristoyl group.

4-6. (canceled)

7. The composition of matter of claim 1, wherein said peptide comprising said amino acid sequence of said NLS of a HER receptor is a stapled peptide, and optionally wherein stapling is effected by replacing amino acids residues i and i+4 of said amino acid sequence with S-pentenylalanine (S5); or by replacing amino acids residues i and i+7 of said amino acid sequence with R-octenylalanine (R8) and S-pentenylalanine.

8. (canceled)

9. A polynucleotide encoding a peptide comprising an amino acid sequence of a nuclear localization sequence (NLS) of a HER receptor capable of binding a nucleolin polypeptide, said amino acid sequence of said NLS does not exceed 16 amino acids.

10. (canceled)

11. The composition of matter of claim 1, wherein said amino acid sequence of said NLS is does not exceed 16 amino acids.

12. (canceled)

13. A nucleic acid construct comprising the polynucleotide of claim 9 and a cis-acting regulatory element for directing expression of said polynucleotide.

14. A host cell comprising the polynucleotide of claim 9.

15. The composition of matter of claim 1, wherein said NLS of said HER receptor is an NLS of epidermal growth factor receptor (EGFR).

16. (canceled)

17. The composition of matter of claim 1, wherein said amino acid sequence is as set forth in SEQ ID NO: 1.

18. (canceled)

19. The composition of matter of claim 1, wherein said amino acid sequence is as set forth in SEQ ID NO: 18, 19, 20, 21 or 22.

20. The composition of matter of claim 1, wherein said NLS of said HER receptor is an NLS of a receptor selected from the group consisting of HER2, HER3 and HER4.

21. (canceled)

22. The composition of matter of claim 1, wherein said amino acid sequence is as set forth in SEQ ID NO: 12, 13 or 14.

23-25. (canceled)

26. A method of treating a disease associated with a HER receptor, the method comprising administering to the subject a therapeutically effective amount of the composition of matter of claim 1, thereby treating the disease in the subject.

27. (canceled)

28. The method of claim 26, wherein pathologic cells of said disease overexpress said HER receptor associated with said disease as compared to non-pathologic cells of the same origin, and/or wherein pathologic cells of said disease overexpress said nucleolin as compared to non-pathologic cells of the same origin, and/or wherein pathologic cells of said disease overexpress Ras as compared to non-diseased cells of the same origin.

29-31. (canceled)

32. The method of claim 26, further comprising administering an additional therapeutic agent specific for a HER receptor, nucleolin and/or Ras.

33-34. (canceled)

35. The method of claim 32, wherein said additional therapeutic agent is GroA.

36. The method of claim 26, wherein said disease is selected from the group consisting of cancer, psoriasis and atherosclerosis.

37-40. (canceled)

41. A method of producing a peptide, the method comprising introducing into a host cell the polynucleotide of claim 9.

42. (canceled)

43. A method of producing a peptide, the method comprising chemically synthesizing the composition of matter of claim 1.

44. A method of producing a peptide, the method comprising chemically synthesizing a peptide comprising an amino acid sequence of a nuclear localization sequence (NLS) of a HER receptor capable of binding a nucleolin polypeptide, said amino acid sequence of said NLS is 16 amino acids long or less.


SEQUENCE LISTING (22 sequences)

SEQ ID NO: 1 - protein, 15 aa, synthetic construct
  REGION 1..15: note = NLS aa sequence of human EGFR or NLS peptide
RRRHIVRKRTLRRLL

SEQ ID NO: 2 - protein, 13 aa, synthetic construct
  REGION 1..13: note = TAT peptide
GRKKRRQRRRPPQ

SEQ ID NO: 3 - protein, 28 aa, synthetic construct
  REGION 1..28: note = TAT-NLS peptide
GRKKRRQRRRPPQRRRHIVRKRTLRRLL

SEQ ID NO: 4 - protein, 20 aa, synthetic construct
  REGION 1..20: note = R-NLS peptide
RRRRRRRRHIVRKRTLRRLL

SEQ ID NO: 5 - protein, 15 aa, synthetic construct
  REGION 1..15: note = Myr-NLS peptide
  SITE 1: note = N-terminally myristoylated
RRRHIVRKRTLRRLL

SEQ ID NO: 6 - protein, 15 aa, synthetic construct
  REGION 1..15: note = myr-NLS-FITC peptide
  SITE 1: note = MISC_FEATURE - N-terminally myristoylated
  SITE 8: note = MISC_FEATURE - Fluorescein isothiocyanate (FITC) labeled
RRRHIVRKRTLRRLL

SEQ ID NO: 7 - protein, 16 aa, synthetic construct
  REGION 1..16: note = mN6-10 peptide
  SITE 1: note = misc_feature - N-terminally myristoylated
  SITE 6: note = misc_feature - Xaa is S-pentenylalanine (S5)
  SITE 10: note = misc_feature - Xaa is S-pentenylalanine (S5)
RRRHIXRKRXTLRRLL

SEQ ID NO: 8 - protein, 15 aa, synthetic construct
  REGION 1..15: note = mN7-11 peptide
  SITE 1: note = misc_feature - N-terminally myristoylated
  SITE 7: note = misc_feature - Xaa is S-pentenylalanine (S5)
  SITE 11: note = misc_feature - Xaa is S-pentenylalanine (S5)
RRRHIVXKRTXRRLL

SEQ ID NO: 9 - protein, 15 aa, synthetic construct
  REGION 1..15: note = mN8-12 peptide
  SITE 1: note = misc_feature - N-terminally myristoylated
  SITE 8: note = misc_feature - Xaa is S-pentenylalanine (S5)
  SITE 12: note = misc_feature - Xaa is S-pentenylalanine (S5)
RRRHIVRXRTLXRLL

SEQ ID NO: 10 - protein, 15 aa, synthetic construct
  REGION 1..15: note = mN11-15 peptide
  SITE 1: note = misc_feature - N-terminally myristoylated
  SITE 11: note = misc_feature - Xaa is S-pentenylalanine (S5)
  SITE 15: note = misc_feature - Xaa is S-pentenylalanine (S5)
RRRHIVRKRTXRRLX

SEQ ID NO: 11 - protein, 15 aa, synthetic construct
  REGION 1..15: note = mN8-15 peptide
  SITE 1: note = misc_feature - N-terminally myristoylated
  SITE 8: note = misc_feature - Xaa is R-octenylalanine (R8)
  SITE 15: note = misc_feature - Xaa is S-pentenylalanine (S5)
RRRHIVRXRTLRRLX

SEQ ID NO: 12 - protein, 16 aa, synthetic construct
  REGION 1..16: note = NLS aa sequence of human HER2
KRRQQKIRKYTMRRLL

SEQ ID NO: 13 - protein, 15 aa, synthetic construct
  REGION 1..15: note = NLS aa sequence of human HER3
RGRRIQNKRAMRRYL

SEQ ID NO: 14 - protein, 15 aa, synthetic construct
  REGION 1..15: note = NLS aa sequence of human HER3
RRKSIKKKRALRRFL

SEQ ID NO: 15 - protein, 16 aa, synthetic construct
  REGION 1..16: note = Myr-NLS2 peptide
  SITE 1: note = MISC_FEATURE - N-terminally myristoylated
KRRQQKIRKYTMRRLL

SEQ ID NO: 16 - protein, 15 aa, synthetic construct
  REGION 1..15: note = Myr-NLS3 peptide
  SITE 1: note = MISC_FEATURE - N-terminally myristoylated
RGRRIQNKRAMRRYL

SEQ ID NO: 17 - protein, 15 aa, synthetic construct
  REGION 1..15: note = Myr-NLS4 peptide
  SITE 1: note = MISC_FEATURE - N-terminally myristoylated
RRKSIKKKRALRRFL

SEQ ID NO: 18 - protein, 16 aa, synthetic construct
  REGION 1..16: note = mN6-10 peptide
  SITE 6: note = misc_feature - Xaa is S-pentenylalanine (S5)
  SITE 10: note = misc_feature - Xaa is S-pentenylalanine (S5)
RRRHIXRKRXTLRRLL

SEQ ID NO: 19 - protein, 15 aa, synthetic construct
  REGION 1..15: note = mN7-11 peptide
  SITE 7: note = misc_feature - Xaa is S-pentenylalanine (S5)
  SITE 11: note = misc_feature - Xaa is S-pentenylalanine (S5)
RRRHIVXKRTXRRLL

SEQ ID NO: 20 - protein, 15 aa, synthetic construct
  REGION 1..15: note = mN8-12 peptide
  SITE 8: note = misc_feature - Xaa is S-pentenylalanine (S5)
  SITE 12: note = misc_feature - Xaa is S-pentenylalanine (S5)
RRRHIVRXRTLXRLL

SEQ ID NO: 21 - protein, 15 aa, synthetic construct
  REGION 1..15: note = mN11-15 peptide
  SITE 11: note = misc_feature - Xaa is S-pentenylalanine (S5)
  SITE 15: note = misc_feature - Xaa is S-pentenylalanine (S5)
RRRHIVRKRTXRRLX

SEQ ID NO: 22 - protein, 15 aa, synthetic construct
  REGION 1..15: note = mN8-15 peptide
  SITE 8: note = misc_feature - Xaa is R-octenylalanine (R8)
  SITE 15: note = misc_feature - Xaa is S-pentenylalanine (S5)
RRRHIVRXRTLRRLX